(19) United States
(12) Patent Application Publication
(10) Pub. No.: US 2024/0181278 A1
(43) Pub. Date: Jun. 6, 2024

(54) APODIZING BACKING STRUCTURES FOR ULTRASONIC TRANSDUCERS AND RELATED METHODS

(71) Applicant: RESONANT ACOUSTICS INTERNATIONAL INC., Markham, Ontario (CA)

(72) Inventors: Nicholas Chris CHAGGARES, Markham, Ontario (CA); Khachic Chris KARSHAFIAN, Markham, Ontario (CA); Eric RIEDER, Markham, Ontario (CA)

(21) Appl. No.: 18/550,308

(22) PCT Filed: Mar. 15, 2022
(86) PCT No.: PCT/CA2022/050387

§ 371 Date: Sep. 13, 2023

Related U.S. Application Data
(60) Provisional application No. 63/200,559, filed on Mar. 15, 2021.

(51) Int. Cl. A61N 7/00; B06B 1/06

(52) U.S. Cl. CPC A61N 7/00; B06B 1/0651; B06B 1/0677; A61N 2007/0095; B06B 2201/76

(57) ABSTRACT

There is provided an apodizing wedge structure for a LIPUS treatment head. The LIPUS treatment head includes a low-volume fraction piezoelectric composite disc. The apodizing wedge structure includes an annular body for contacting a surface of the piezoelectric disc, the annular body including an inner perimeter having an inner thickness and an outer perimeter having an outer thickness. The annular body includes an inclined surface forming a continuous slope extending from the inner perimeter to the outer perimeter, the inner thickness being smaller than the outer thickness. The apodizing wedge is configured to change an apparent thickness of the piezoelectric disc with respect to resonant properties of the piezoelectric disc when the apodizing wedge structure is in acoustic communication with the piezoelectric disc, thereby allowing the LIPUS treatment head to generate a uniform near field. LIPUS treatment heads and ultrasonic transducers including such an apodizing wedge structure are also provided.

### TECHNICAL FIELD

The technical field generally relates to the field of acoustic energy and more particularly relates to an apodizing backing structure for ultrasonic transducers, related devices, apparatuses, methods and techniques.

### BACKGROUND

Ultrasonic transducers are widely used in many industries and for a broad variety of applications. For example, ultrasonic transducers can be employed in medical applications, including diagnostic imaging or therapeutic applications. Other applications include but are not limited to ultrasonic non-destructive testing and ultrasonic machining and welding. Ultrasonic transducers can be configured to change electrical energy into mechanical energy, convert acoustic energy into electrical energy, or they can be configured to do both reciprocally.

There is still a need for techniques, apparatus, devices, and methods that alleviate or mitigate the problems of prior art.

### SUMMARY

The present techniques generally concern a low-Intensity pulsed ultrasound (LIPUS) treatment head, and more specifically relate to an apodizing backing structure for LIPUS treatment head configured to generate a substantially uniform near field or to generate an acoustic field including at least one substantially uniform near field component or portion.

In accordance with one aspect, there is provided an ultrasonic transducer, including:
a low-volume fraction piezoelectric composite disc having resonant properties:
at least one electrode in electrical contact with the low-volume fraction piezoelectric composite disc: and
an annular apodizing backing structure in acoustic contact with the low-volume fraction piezoelectric composite disc, the annular apodizing backing structure having:
   an inner perimeter and a corresponding inner thickness;
   an outer perimeter and a corresponding outer thickness; and
   an inclined surface forming a substantially continuous slope extending from the inner perimeter to the outer perimeter, the inner thickness being smaller than the outer thickness,
      wherein the annular apodizing backing structure is configured to change an apparent thickness of the low-volume fraction piezoelectric composite disc with respect to the resonant properties of the low-volume fraction piezoelectric composite disc, thereby allowing the ultrasonic transducer to generate an acoustic field including at least one substantially uniform near field component or portion.

In some embodiments, the ultrasonic transducer further includes a circuit board. In some embodiments, the circuit board is a printed circuit board. In some embodiments, the printed circuit board is a ring-shaped printed circuit board.

In some embodiments, the low-volume fraction piezoelectric composite disc is in a 1 3 configuration.

In some embodiments, the low-volume fraction piezoelectric composite disc includes 280 μm by 280 μm pillars distributed in a 2D matrix pattern having a pitch of about 480 μm in both lateral axes.

In some embodiments, the low-volume fraction piezoelectric composite disc is configured to operate in a half-wave resonant mode at 1.5 MHz.

In some embodiments, the low-volume fraction piezoelectric composite disc has an acoustic impedance included in a range extending from about 9 MR to about 13 MR.

In some embodiments, the acoustic impedance is about 11 MR.

In some embodiments, the low-volume fraction piezoelectric composite disc has a thickness of about λ/2 at 1.5 MHz.

In some embodiments, the low-volume fraction piezoelectric composite disc includes a lead zirconate titanate material (PZT) based material.

In some embodiments, the PZT-based material is PZT 5H.

In some embodiments, the low-volume fraction piezoelectric composite disc includes about 35% of PZT 5H and about 65% of a polymer matrix.

In some embodiments, the polymer matrix includes epoxy filled with micro glass balloons and silicone particles.

In some embodiments, the PZT 5H pillars have a first bar-mode longitudinal acoustic velocity and the polymer matrix has a second longitudinal acoustic velocity, the second longitudinal velocity being approximately 60% to 70% of the first longitudinal velocity.

In some embodiments, the first longitudinal bar-mode acoustic velocity is about 3850 m/s and the second longitudinal acoustic velocity is about 2515 m/s.

In some embodiments, the ultrasonic transducer further comprises ring-shaped printed circuit board having an inner diameter; and the low-volume fraction piezoelectric composite disc has an outer diameter, the inner diameter of the ring-shaped printed circuit board being larger than the outside diameter of the low-volume fraction piezoelectric composite disc.

In some embodiments, the ultrasonic transducer further includes a housing, the housing being made from plastic.

In some embodiments, the housing includes a matching layer in acoustic communication with the low-volume fraction piezoelectric composite disc.

In some embodiments, the matching layer has a thickness of about λ/4.

In some embodiments, the matching layer is integrally formed with the housing.

In some embodiments, the matching layer has an acoustic impedance included in a range extending from about 2.1 MR to about 2.5 MR.

In some embodiments, the acoustic impedance is about 2.3 MR.

In some embodiments, the ultrasonic transducer further includes a 2λ/3 acoustic layer in acoustic communication with the low-volume fraction piezoelectric composite disc.

In some embodiments, the 2λ/3 acoustic layer is made from nonyl plastic and has a thickness of about 953 μm.

In some embodiments, the ultrasonic transducer further includes a 0.9λ acoustic layer, in acoustic communication with the low-volume fraction piezoelectric composite disc.

In some embodiments, the 0.9λ acoustic layer is made from nonyl plastic and has a thickness of about 1.28 mm.

In some embodiments, the ring-shaped printed circuit board included two opposed planar surfaces, each planar surface being made from copper.

In some embodiments, the ring-shaped printed circuit board is bonded with a perimeter of the low-volume fraction piezoelectric composite disc.

In some embodiments, the ultrasonic transducer further includes an inductor connected in parallel with a piezocomposite of the low-volume fraction piezoelectric composite disc, the inductor being configured to resonate with the low-volume fraction piezoelectric composite disc, the annular apodizing backing structure, and the ¼ lambda acoustic matching layer, such that an impedance maximum is produced at about 1.5 MHz when a distal face of the transducer is air loaded.

In some embodiments, the ultrasonic transducer further includes an inductor connected in series with a low-volume fraction piezoelectric composite disc, the inductor being configured to resonate with the low-volume fraction piezoelectric composite disc the annular apodizing backing structure, and the ¼ lambda matching layer, such that an impedance minimum is produced at about 1.5 MHz when a distal face of the transducer is air loaded.

In some embodiments, the annular apodizing backing structure includes Epotek 301 epoxy.

In some embodiments, the annular apodizing backing structure has an acoustic impedance of about 2.8 MR. In some embodiments, the slope is included between 0 degrees and 30 degrees.

In some embodiments, the slope is about 14 degrees with respect to a top surface of the low-volume fraction piezoelectric composite disc.

In some embodiments, the ultrasonic transducer is operable at a frequency of about 1.5 MHz.

In some embodiments, the ultrasonic transducer is operable in a narrow bandwidth tone burst mode.

In some embodiments, the narrow bandwidth tone burst mode is a 20% duty cycle sinusoidal pulsed mode, preferably at a pulse repetition frequency of about 1 kHz.

In some embodiments, the ultrasonic transducer has a beam non-uniformity ratio of less than 3.5.

In some embodiments, the at least one substantially uniform near field component or portion exhibits less than 2 dB of ripples in a plane located at about 3 mm of an external surface of the ultrasonic transducer, when the ultrasonic transducer is operated at 1.5 MHz with a 20% pulsed transmit waveform.

In accordance with another aspect, there is provided a low-intensity pulsed ultrasound (LIPUS) treatment head having an operating frequency, the LIPUS treatment head including:
an acoustic stack, including:
   a piezoelectric disc, the piezoelectric disc including a low-volume fraction piezoelectric composite disc, the low-volume fraction piezoelectric composite disc being configured to operate in a half-wave resonant mode at the operating frequency of the LIPUS treatment head; and
   an annular apodizing backing structure in acoustic communication with the low-volume fraction piezoelectric composite disc, the annular apodizing backing structure having an inner perimeter and an outer perimeter, respectively having an inner thickness and an outer thickness, the inner thickness being smaller than the outer thickness, the annular apodizing backing structure being configured to change an apparent thickness of the low-volume fraction piezoelectric composite disc with respect to the resonant properties of the low-volume fraction piezoelectric composite disc, thereby allowing the LIPUS treatment head to generate an acoustic field including at least one substantially uniform near field or portion;
at least one electrode in electrical communication with the low-volume fraction piezoelectric composite disc; and
a housing for supporting the acoustic stack and the at least one electrode.

In some embodiments, the LIPUS treatment head further includes a circuit board. In some embodiments, the circuit board is a printed circuit board. In some embodiments, the printed circuit board is a ring-shaped printed circuit board.

In some embodiments, the low-volume fraction piezoelectric composite disc is in a 1 3 configuration.

In some embodiments, the low-volume fraction piezoelectric composite disc includes 280 μm by 280 μm pillars distributed in a 2D matrix pattern having a pitch of about 480 μm in both lateral axes.

In some embodiments, the operating frequency of the LIPUS treatment head is about 1.5 MHz.

In some embodiments, the low-volume fraction piezoelectric composite disc has an acoustic impedance included in a range extending from about 9 MR to about 13 MR.

In some embodiments, the acoustic impedance is about 11 MR.

In some embodiments, the low-volume fraction piezoelectric composite disc has a thickness of about 2 at 1.5 MHz.

In some embodiments, the low-volume fraction piezoelectric composite disc includes a lead zirconate titanate material (PZT) based material.

In some embodiments, the PZT-based material is PZT 5H.

In some embodiments, wherein the low-volume fraction piezoelectric composite disc includes about 35% of PZT 5H and about 65% of a polymer matrix.

In some embodiments, the polymer matrix includes epoxy filled with micro glass balloons and silicone particles.

In some embodiments, the PZT 5H pillars have a first bar-mode longitudinal acoustic velocity and the polymer matrix has a second longitudinal acoustic velocity, the second longitudinal velocity being approximately 60 to 70% of the first longitudinal velocity.

In some embodiments, the first longitudinal acoustic velocity is about 3850 m/s and the second longitudinal acoustic velocity is about 2515 m/s.

In some embodiments, the ring-shaped printed circuit board has an inner diameter; and the low-volume fraction piezoelectric composite disc has an outer diameter, the inner diameter of the ring-shaped printed circuit board being larger than the outside diameter of the low-volume fraction piezoelectric composite disc. In some embodiments, the housing is made from plastic.

In some embodiments, the housing includes a matching layer in acoustic communication with the low-volume fraction piezoelectric composite disc.

In some embodiments, the matching layer has a thickness of about λ/4.

In some embodiments, the matching layer is integrally formed with the housing.

In some embodiments, the matching layer has acoustic impedance included in a range extending from about 2.1 MR to about 2.5 MR.

In some embodiments, the acoustic impedance is about 2.3 MR.

In some embodiments, the LIPUS treatment head further includes a 2λ/3 acoustic layer in acoustic communication with the low-volume fraction piezoelectric composite disc.

In some embodiments, the 2λ/3 acoustic layer is made from nonyl plastic and has a thickness of about 953 μm.

In some embodiments, the LIPUS treatment head further includes a 0.9λ acoustic layer, in acoustic communication with the low-volume fraction piezoelectric composite disc.

In some embodiments, the 0.9λ acoustic layer is made from nonyl plastic and has a thickness of about 1.28 mm.

In some embodiments, the ring-shaped printed circuit board included two opposed planar surfaces, each planar surface being made from copper.

In some embodiments, the ring-shaped printed circuit board is bonded with a perimeter of the low-volume fraction piezoelectric composite disc.

In some embodiments, the LIPUS treatment head further includes an inductor connected in parallel with a piezocomposite of the low-volume fraction piezoelectric composite disc, the inductor being configured to resonate with the low-volume fraction piezoelectric composite disc, the annular apodizing backing structure, and the ¼ lambda matching layer, such that an impedance maximum is produced at approximately 1.5 MHz when a distal face of the transducer is air loaded.

In some embodiments, the LIPUS treatment head further includes an inductor connected in series with a piezocomposite of the low-volume fraction piezoelectric composite disc, the inductor being configured to resonate with the low-volume fraction piezoelectric composite disc, the annular apodizing backing structure, and the ¼ lambda matching layer, such that an impedance minimum is produced at approximately 1.5 MHz when the distal face of the transducer is air loaded.

In some embodiments, the annular apodizing backing structure includes Epotek 301 epoxy.

In some embodiments, the annular apodizing backing structure has an acoustic impedance of about 2.8 MR.

In some embodiments, the slope is included between 0 to 30 degrees.

In some embodiments, the slope is about 14 degrees with respect to a top surface of the low-volume fraction piezoelectric composite disc.

In some embodiments, the operating frequency of LIPUS treatment head is about 1.5 MHz.

In some embodiments, the LIPUS treatment head is operable in a narrow bandwidth tone burst mode.

In some embodiments, the narrow bandwidth tone burst mode is a 20% duty cycle sinusoidal pulsed mode.

In some embodiments, wherein the LIPUS treatment head has beam non-uniformity ratio of less than 3.5.

In some embodiments, the at least one substantially uniform near component or portion field exhibits less than 2 dB of ripple in a plane located at about 3 mm of an external surface of the LIPUS treatment head, when the LIPUS treatment head is operated at 1.5 MHz with a 20% pulsed transmit waveform.

In accordance with another aspect, there is provided an apodizing wedge structure for a low-intensity pulsed ultrasound (LIPUS) treatment head, the LIPUS treatment head including a low-volume fraction piezoelectric composite disc, the apodizing wedge structure including:
an annular body for contacting a surface of the low-volume fraction piezoelectric composite disc, the annular body including an inner perimeter having a corresponding inner thickness and an outer perimeter having a corresponding outer thickness,
   wherein the annular body includes an inclined surface forming a substantially continuous slope extending from the inner perimeter to the outer perimeter, the inner thickness being smaller than the outer thickness, the apodizing wedge being configured to change an apparent thickness of the low-volume fraction piezoelectric composite disc with respect to resonant properties of the low-volume fraction piezoelectric composite disc when the apodizing wedge structure is in acoustic communication with the low-volume fraction piezoelectric composite disc, thereby allowing the LIPUS treatment head to generate an acoustic field including at least one substantially uniform near field component or portion.

In some embodiments, the annular body is made from Epotek 301 epoxy.

In some embodiments, the annular body has an acoustic impedance of about 2.8 MR.

In some embodiments, the slope is included between 0 to 30 degrees.

In some embodiments, the slope is about 14 degrees with respect to a top surface of the low-volume fraction piezoelectric composite disc.

In some embodiments, the LIPUS treatment head is operable at a frequency of about 1.5 MHz.

In some embodiments, the LIPUS treatment head is operable in a narrow bandwidth tone burst mode.

In some embodiments, the narrow bandwidth tone burst mode is a 20% duty cycle sinusoidal pulsed mode.

In some embodiments, the LIPUS treatment head has a beam non-uniformity ratio of less than 3.5.

In some embodiments, the at least one substantially uniform near field component or portion exhibits less than 2 dB of ripples in a plane located at about 3 mm of an external surface of the LIPUS treatment head, when the LIPUS treatment head is operated at 1.5 MHz with a 20% pulsed transmit waveform.

In accordance with another aspect, there is provided a backing structure for a low-intensity pulsed ultrasound (LIPUS) treatment head, the LIPUS treatment head including a low-volume fraction piezoelectric composite element, the backing structure including:
a body for contacting a surface of the low-volume fraction piezoelectric composite element, such that when the body contacts the low-volume fraction piezoelectric composite element, destructive interference is produced within the backing structure and the low-volume fraction piezoelectric component, thereby shaping an acoustic field generated by the LIPUS treatment head, the destructive interference being dependent on a thickness of the backing structure.

In some embodiments, the destructive interference results in a maximal attenuation at approximately λ/4 or odd multiples thereof.

In some embodiments, the body is made from Epotek 301 epoxy.

In some embodiments, the LIPUS treatment head is operable at a frequency of about 1.5 MHz.

In some embodiments, the LIPUS treatment head is operable in a narrow bandwidth tone burst mode.

In some embodiments, the narrow bandwidth tone burst mode is a 20% duty cycle sinusoidal pulsed mode.

In some embodiments, the LIPUS treatment head has a beam non-uniformity ratio of less than 3.5.

In some embodiments, the acoustic field includes at least one substantially uniform near field component or portion, said at least one substantially uniform near field portion exhibiting less than 2 dB of ripples in a plane located at about 3 mm of an external surface of the LIPUS treatment head, when the LIPUS treatment head is operated at 1.5 MHz with a 20% pulsed transmit waveform.

In accordance with another aspect, there is provided a method of apodizing an acoustic field, the method including:
operating an ultrasonic transducer to generate the acoustic field, the ultrasonic transducer including a low-volume fraction piezoelectric composite disc, the low-volume fraction piezoelectric composite having resonant properties; and conditioning the acoustic field with an annular apodizing backing structure to generate an apodized acoustic field, the apodized acoustic field including at least one substantially uniform near field component or portion, the annular apodizing backing structure being in acoustic contact with the low-volume fraction piezoelectric composite disc, the annular apodizing backing structure having:
an inner perimeter and a corresponding inner thickness;
an outer perimeter and a corresponding outer thickness; and
an inclined surface forming a substantially continuous slope extending from the inner perimeter to the outer perimeter, the inner thickness being smaller than the outer thickness,
   wherein the annular apodizing backing structure is configured to change an apparent thickness of the low-volume fraction piezoelectric composite disc with respect to the resonant properties of the low-volume fraction piezoelectric composite disc.

In some embodiments:
the ultrasonic transducer is operated at 1.5 MHz with a 20% pulsed transmit waveform; and
the at least one substantially uniform near field component or portion exhibits less than 2 dB of ripples in a plane located at about 3 mm of an external surface of the ultrasonic transducer.

In some embodiments, the low-volume fraction piezoelectric composite disc is in a 1 3 configuration.

In some embodiments, the low-volume fraction piezoelectric composite disc includes 280 μm by 280 μm pillars distributed in a 2D matrix pattern having a pitch of about 480 μm in both lateral axes.

In accordance with another aspect, there is provided a method for generating an acoustic field with a low-intensity pulsed ultrasound (LIPUS) treatment head having an operating frequency, the method including:
operating the LIPUS treatment to generate the acoustic field, the LIPUS treatment head including:
   an acoustic stack, the acoustic stack including:
      a piezoelectric disc, the piezoelectric disc including a low-volume fraction piezoelectric composite disc, the low-volume fraction piezoelectric composite disc being configured to operate in a half-wave resonant mode at the operating frequency of the LIPUS treatment head; and
      an annular apodizing backing structure in acoustic communication with the low-volume fraction piezoelectric composite disc, the annular apodizing backing structure having an inner perimeter and an outer perimeter, respectively having an inner thickness and an outer thickness, the inner thickness being smaller than the outer thickness;
   at least one electrode in electrical communication with the low-volume fraction piezoelectric composite disc; and
   a housing for supporting the acoustic stack and the at least one electrode; and
conditioning the acoustic field with the annular apodizing backing structure to generate an apodized acoustic field, the apodized acoustic field including at least one substantially uniform near field component or portion.

In some embodiments, the LIPUS treatment head further includes a circuit board. In some embodiments, the circuit board is a printed circuit board. In some embodiments, the printed circuit board is a ring-shaped printed circuit board.

In some embodiments:
the ultrasonic transducer is operated at 1.5 MHz with a 20% pulsed transmit waveform; and
the at least one substantially uniform near field component or portion exhibits less than 2 dB of ripples in a plane located at about 3 mm of an external surface of the ultrasonic transducer.

In some embodiments, the low-volume fraction piezoelectric composite disc is in a 1 3 configuration.

In some embodiments, the low-volume fraction piezoelectric composite disc includes 280 μm by 280 μm pillars distributed in a 2D matrix pattern having a pitch of about 480 μm in both lateral axes.

Other features and advantages of the present description will become more apparent upon reading of the following non-restrictive description of specific embodiments thereof, given by way of example only with reference to the accompanying drawings.

### BRIEF DESCRIPTION OF THE DRAWINGS

FIG. 1 illustrates an ultrasonic transducer, in accordance with one embodiment.

FIG. 2 illustrates an exploded view of the ultrasonic transducer shown in FIG. 1.

FIG. 3 illustrates an exploded view of the ultrasonic transducer shown in FIG. 1.

FIG. 4 is a cross section perspective view of a low-volume fraction piezoelectric composite disc in acoustic communication with an annular backing structure, in accordance with one embodiment.

FIG. 5 is another cross section perspective view the low-volume fraction piezoelectric composite disc in acoustic communication with the annular backing structure illustrated in FIG. 4.

FIG. 6 is an exploded view of a of a low-volume fraction piezoelectric composite disc and an annular backing structure, in accordance with one embodiment.

FIG. 7 is another view the low-volume fraction piezoelectric composite disc and the PSPCB structure illustrated in FIG. 6.

FIG. 8 is another view the low-volume fraction piezoelectric composite disc and annular backing structure illustrated in FIG. 6.

FIG. 9A and FIG. 9B illustrate a linear graph of the pressure magnitude response of a conventional ultrasonic transducer (left portion) and a linear graph of the pressure magnitude response of an ultrasonic transducer designed according to the present techniques (right portion).

FIGS. 10A-D show a simulated field showing outer maximum ring present in a 3 mm field of a 1 3 composite-based LIPUS treatment head with no apodization ring or structure (top left portion); a measured acoustic field showing outer maximum ring present in the 3 mm field of the 1 3 composite-based LIPUS treatment head (top right portion); a simulated field showing suppressed outer maximum ring resulting from the inclusion of an apodizing wedge (or an “annular apodizing backing structure) on a perimeter portion of the back surface of the 1 3 composite-based disc (bottom left portion); and a measured acoustic field from a prototype LIPUS treatment head showing suppressed outer maximum ring achieved with the apodizing ring (or annular apodizing backing structure) on the 1 3 composite-based disc (bottom right portion).

FIGS. 11A,B show the intensity measured at the 3 mm plane and the intensity measured at the plane containing the last axial maximum of a LIPUS treatment head incorporating the present techniques.

FIGS. 12A and 12B illustrate the effect of air loading with no inductor (FIG. 12A) compared to air loading with a parallel inductor configured to resonate at 1.5 MHz (FIG. 12B) of the present technology, with parallel inductive resonance tuned to match operating frequency when the ultrasonic transducer is air loaded

FIG. 13A shows the impedance of the nonlimitative embodiment of the LIPUS treatment head having a λ/4 matching layer and a resonating parallel inductor with air and water loading. FIG. 13B shows the impedance of the nonlimitative embodiment of the LIPUS treatment head Exemplary transducer having a λ/4 matching layer but no resonating inductor, with air and water loading cases.

FIG. 14 is a 3D plot of an apodized acoustic field generated with the present techniques, measured in water tank with hydrophone at the plane located 3 mm from the distal face of the treatment head.

FIGS. 15A,B show a 2λ/3 front layer tuned to provide impedance maxima (shown in the bottom half of the figure) at operating frequency in air without the use of a resonant inductor in the circuit, compared to the response in water (shown in the top half of the figure), the air coupled maximum being approximately 5 times higher than the water coupled minimum.

FIGS. 16A,B shows a 0.9λ front layer tuned to provide an impedance minimum in air (shown in the bottom half of the figure), and a higher impedance when water coupled (shown in the top portion of the figure), the air-coupled impedance minimum being approximately 3 times lower than that when water coupled (bottom portion).

FIGS. 17A,B show the axial pressure response of the ultrasonic transducer, according to the present techniques. FIG. 17A illustrates a highly uniform on-axis pressure field in the first 10 cm of the near field, and an absence of high amplitude peaks in the entire near field. Figure B illustrates the lack of near field axial uniformity seen in the absence of the apodization backing structure and corresponding acoustic field.

FIG. 18 illustrates the localized attenuation of a transmitted acoustic field due to destructive interference generated by the presence of the backing structure versus the thickness of the backing structure as a fraction of the wavelength.

FIGS. 19A,B are representative of a method of achieving a difference in impedance at the working frequency of the device, the air impedance magnitude being several times lower than the water impedance and is achieved using a series resonant inductor, this arrangement produces a result similar to that seen in the 0.9λ matching layer case but using a series resonant inductor and λ/4 matching layer.

FIGS. 20A-C present cross section perspective views of an ultrasonic transducer, in accordance with another embodiment.

### DETAILED DESCRIPTION

In the following description, similar features in the drawings have been given similar reference numerals, and, to not unduly encumber the figures, some elements may not be indicated on some figures if they were already identified in one or more preceding figures. It should also be understood herein that the elements of the drawings are not necessarily depicted to scale, since emphasis is placed upon clearly illustrating the elements and structures of the present embodiments. The terms “a”, “an” and “one” are defined herein to mean “at least one”, that is, these terms do not exclude a plural number of elements, unless stated otherwise. It should also be noted that terms such as “substantially”, “generally” and “about”, that modify a value, condition or characteristic of a feature of an exemplary embodiment, should be understood to mean that the value, condition or characteristic is defined within tolerances that are acceptable for the proper operation of this exemplary embodiment for its intended application.

In the present description, the terms “connected”, “coupled”, and variants and derivatives thereof, refer to any connection or coupling, either direct or indirect, between two or more elements. The connection or coupling between the elements may be acoustical, mechanical, physical, optical, operational, electrical, wireless, or a combination thereof.

The terms “match”, “matching” and “matched” are intended to refer herein to a condition in which two elements are either the same or within some predetermined tolerance of each other. That is, these terms are meant to encompass not only “exactly” or “identically” matching the two elements but also “substantially”, “approximately” or “subjectively” matching the two elements, as well as providing a higher or best match among a plurality of matching possibilities.

In the present description, the expression “based on” is intended to mean “based at least partly on”, that is, this expression can mean “based solely on” or “based partially on”, and so should not be interpreted in a limited manner. More particularly, the expression “based on” could also be understood as meaning “depending on”, “representative of”, “indicative of”, “associated with” or similar expressions.

It will be appreciated that positional descriptors indicating the position or orientation of one element with respect to another element are used herein for ease and clarity of description and should, unless otherwise indicated, be taken in the context of the figures and should not be considered limiting. It will be understood that spatially relative terms (e.g., “outer” and “inner”, “outside” and “inside”, “periphery” and “central”, “over” and “under”, and “top” and “bottom”) are intended to encompass different positions and orientations in use or operation of the present embodiments, in addition to the positions and orientations exemplified in the figures.

The description generally relates to an ultrasonic transducer assembly and more particularly concerns a LIPUS treatment head configured to generate a substantially uniform near field (or an acoustic field including at least one substantially uniform near field component or portion), as well as related methods. The technology and its advantages will become more apparent from the detailed description and examples that follow, which describe the various embodiments of the technology. More specifically. the following description will present a LIPUS treatment head that may be used for therapeutic applications. Therapeutic applications include but are not limited to the treatment of biological tissue(s), bone(s), cartilage(s), tendon(s), and the like. For instance, the present techniques may be used to treat tissue(s) injuries or support bone(s) healing.

In the context of the current disclosure, the expressions “apodizing”, “apodization”, “apodized”, synonyms and derivatives thereof refer to techniques that may be used to change. alter, or shape an intensity profile of a field. such as. for example and without being limitative a spatial profile of an acoustic field. In some embodiments. the apodization techniques may be used to spatially attenuate an acoustic field at its edges or along its “perimeter”, or at least portion(s) thereof. Of note, the present technology allows obtaining or producing an apodized acoustic field using an apodizing structure or a backing structure, the apodizing or backing structure being provided at a back portion of the ultrasonic transducer and by taking advantage of destructive interferences generated therein, without relying on techniques for absorbing energy.

While the embodiments of the ultrasonic transducer that will be described throughout the description will be described as including a piezoelectric material, one skilled in the art would note that the ultrasonic transducers of the current disclosure may instead include any ferroelectric materials, any single crystals or polycrystalline materials, any electromechanical transduction materials, such materials having one or more of the following properties: ferroelectricity, pyroelectricity, piezoelectricity, electrostriction and/other relevant properties. It will be noted that, in the context of the present description, the expression “piezoelectric material” may also refer to ferroelectric material, pyroelectric material, relaxor material and electrostrictive material, as it would be readily understood by one skilled in the art.

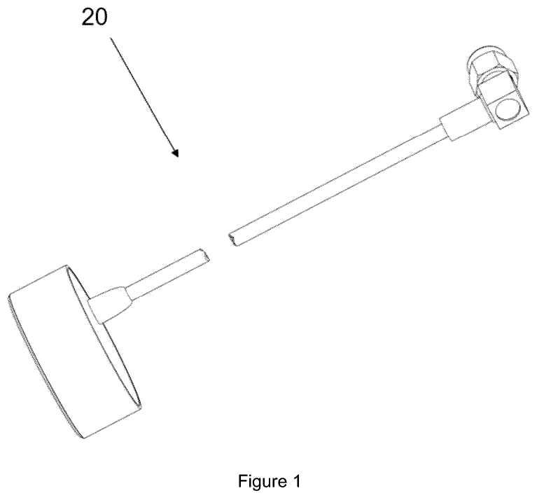

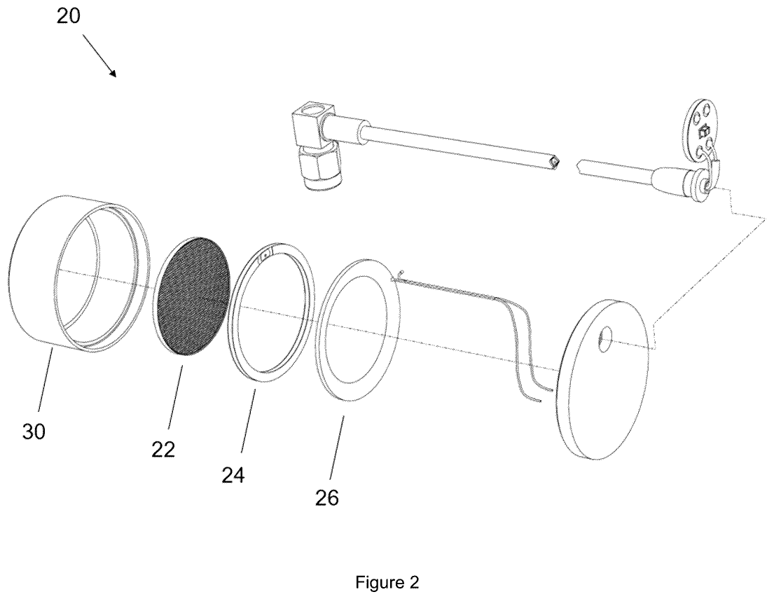

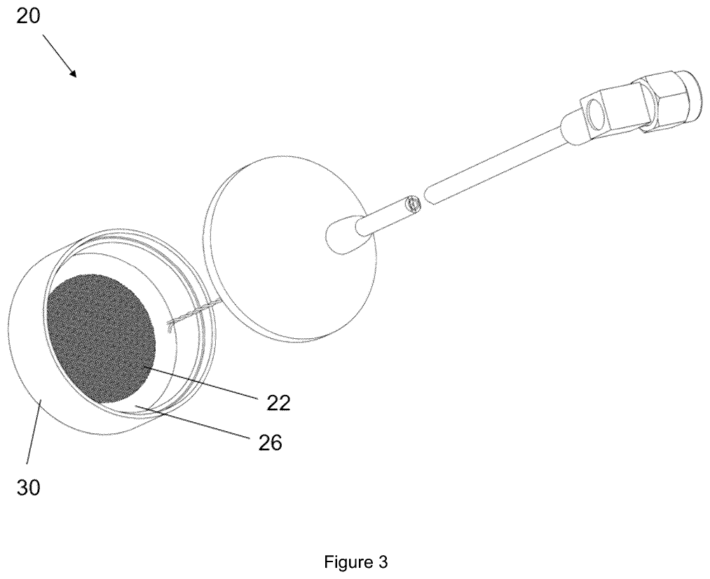

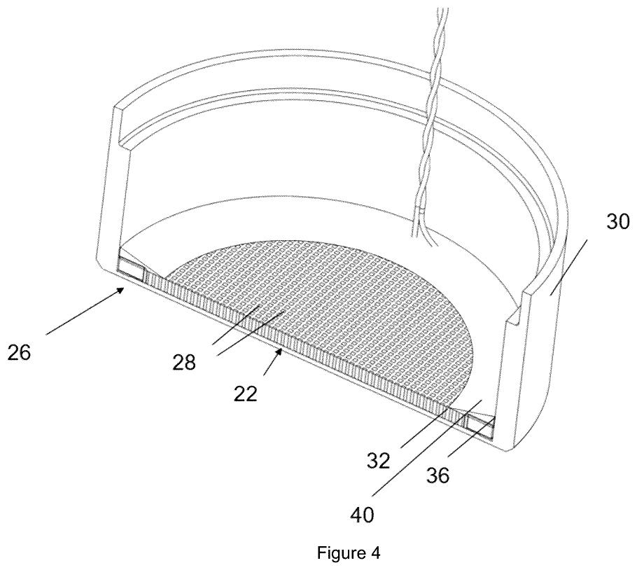

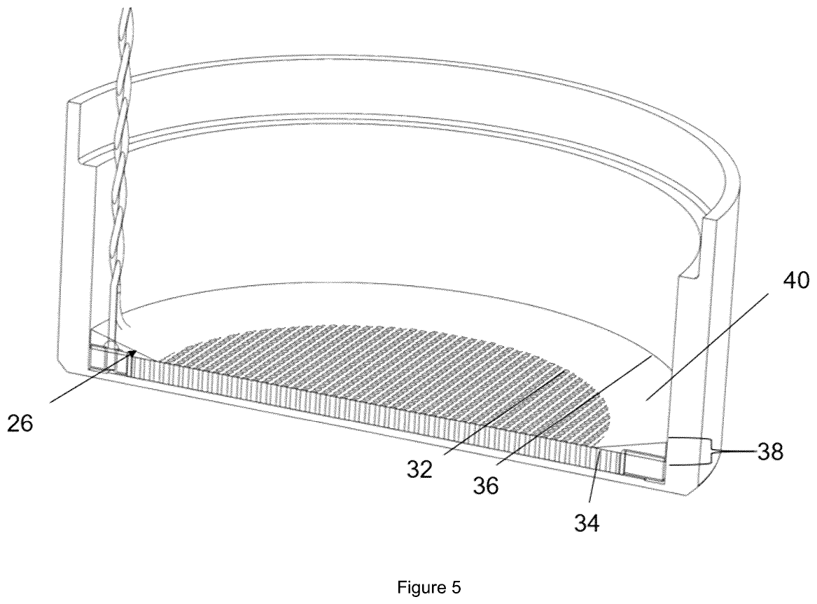

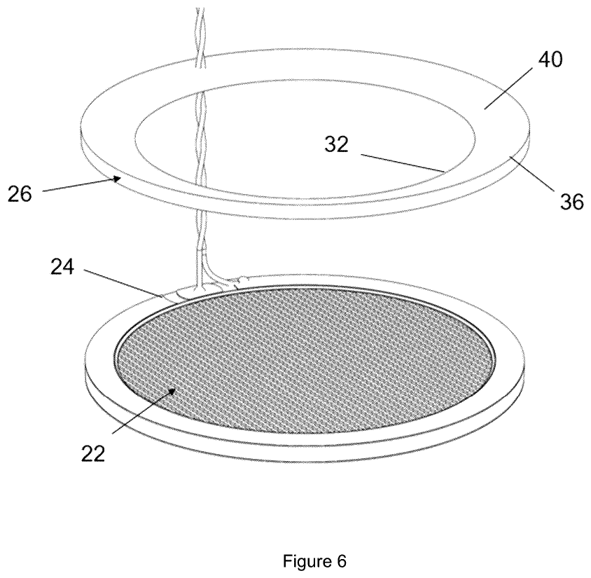

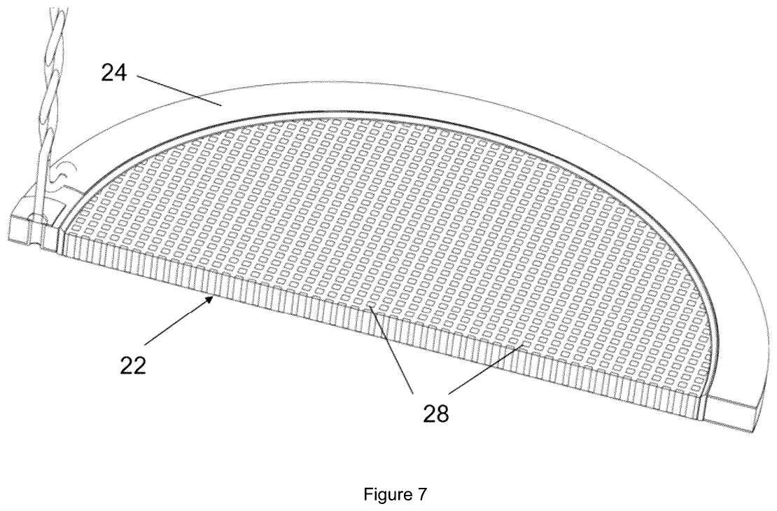

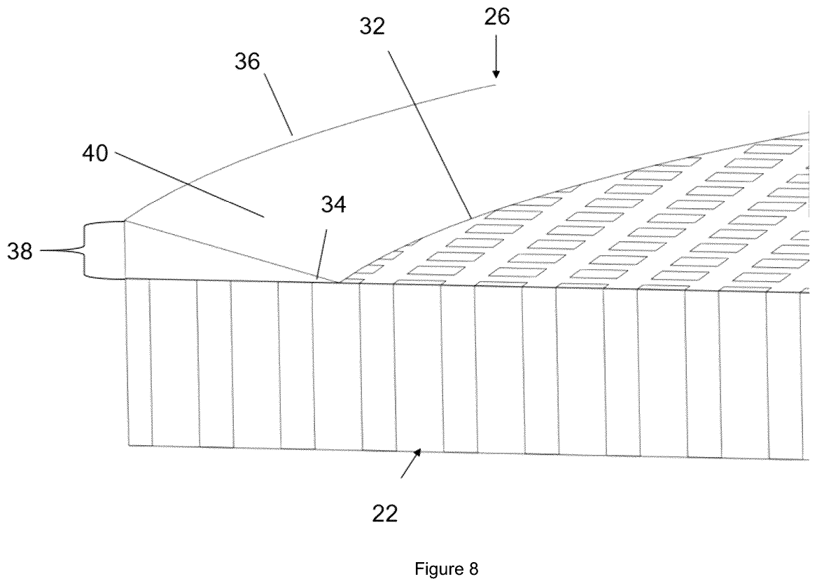

In accordance with one aspect, and with reference to FIGS. 1 to 19A,B, there is provided an ultrasonic transducer 20. Broadly described, the ultrasonic transducer 20 includes a low-volume fraction piezoelectric composite disc 22 having resonant properties, at least one electrode (which may be embodied or replaced by a ring-shaped printed circuit board 24) and an annular backing structure 26 configured to provide attenuation of the acoustic field generated by the low-volume fraction piezoelectric transducer disc 22, based on localized destructive interference. In some embodiments, the ultrasonic transducer 20 may include a low-volume fraction piezoelectric composite element instead of a disc. In these embodiments, the low-volume fraction piezoelectric composite element may be embodied by various plate structures and shapes such as, for example and without being limitative, rectangles, annuli, or curved piezoelectric structures (e.g., a curved focused composite plate). The annular apodizing backing structure 26 is configured to change an apparent thickness of the low-volume fraction piezoelectric composite disc 22 with respect to the resonant properties of the low-volume fraction piezoelectric composite disc 22, thereby allowing the ultrasonic transducer 20 to generate a substantially uniform near field, or an acoustic field including at least one substantially uniform near field component or portion. The interaction between the low-volume fraction piezoelectric composite disc 22, the annular apodizing backing structure 26 and other components of the ultrasonic transducer 20 (e.g., electrodes) allows providing an effective change in the acoustic thickness (i.e., an “apparent” thickness) of the low-volume fraction piezoelectric composite disc 22, without changing the actual thickness (i.e., the “real” or “physical” thickness) of the low-volume fraction piezoelectric composite disc 22. It also allows providing an effective change in the acoustic thickness (i.e., the “apparent” thickness) of the low-volume fraction piezoelectric composite disc 22 without substantially changing the electrical impedance of the low-volume fraction piezoelectric composite disc 22. It is therefore possible to change the resonant frequency of the low-volume piezoelectric disc 22 without changing the wavelength or electroacoustic frequency of the wave produced by the piezoelectric material forming the low-volume fraction piezoelectric composite disc 22, shifting the resonant characteristics of the low-volume fraction piezoelectric composite disc 22, but not the electroacoustic frequency response of the low-volume fraction piezoelectric composite disc 22 itself. As the thickness of the apodizing backing structure 26 approaches ¼ lambda or odd multiples of ¼ lambda (e.g., ¾ lambda or 5/4 lambda), strong destructive interference is created within the low-volume fraction piezocomposite disc 22 and the backing structure 26, resulting in attenuation of the acoustic field in the region of the backing structure 26. In some embodiments, the backing structure 26 may be a wedge located at the perimeter of the low-volume fraction piezoelectric composite disc 22 and can act as an apodizing backing structure. In some embodiments, the ultrasonic transducer 20 is configured to operate in relatively narrow band mode, as the effect of the annular apodizing backing structure 26 has a relatively strong effect in narrow band modes of operation, including, for example and without being limitative, tone bursts or CW. In some embodiments, the annular apodizing backing structure 26 makes it possible to reduce the transmitted acoustic output of the low-volume fraction piezoelectric composite disc 22 in the region of the annular apodizing structure 26 by over 25 dB. The ultrasonic transducer 20 would behave similarly in a receiver mode. The effect of the annular apodizing backing structure 26 may have a limited attenuation impact on broad band modes of operation, since the change of phase produced by the apodizing backing structure 26 can result in destructive interference within the in low-volume fraction piezoelectric composite disc 22 only when a sufficient number of cycles are present within the pulse to interfere within the disc. This technology may become relatively effective when there are more than about 5 cycles, however, in some applications, it may be beneficial with even a very short single cycle or impulse type waveform. The annular apodizing backing structure 26 allows the attenuation and shaping of the edges of the acoustic field, generally producing a smooth transition from the peak values of the acoustic field and the edges of the acoustic field. This change of the field edges reduces side lobes, reduces lateral modes within the piezo elements, and improves uniformity of the edges of the beam. The present techniques for modifying the edges and properties of the acoustic field and thus smoothing and potentially shaping the perimeter of the acoustic field is referred to herein as the apodization of the acoustic field that would typically be generated using the low-volume fraction piezoelectric composite disc 22 alone, i.e., the production of an apodized acoustic field. It should be noted that this destructive interference based backing structure 26 can also be used for general beam shaping and not only for apodizing the perimeter of the field.

Now that the ultrasonic transducer 20 has been broadly described, different embodiments of the low-volume fraction piezoelectric composite disc 22, the ring-shaped printed circuit 24 and the annular apodizing backing structure 26 will be presented.

In some embodiments, the low-volume fraction piezoelectric composite disc 22 may include square pillars 28. For example, and without being limitative, the dimensions of the square pillars 28 may be about 280 μm by about 280 μm for each pillar 28. The square pillars 28 may be distributed in a 2D matrix pattern having a pitch of about 480 μm in both lateral axes. Of note, the lateral axes extend in a plane parallel to one surface of low-volume fraction piezoelectric composite disc 22, i.e., each lateral axis is parallel to a corresponding one of the radius or diameter of the low-volume fraction piezoelectric composite disc 22.

The ultrasonic transducer 20 is generally configured to operate at an operating frequency, and the low-volume fraction piezoelectric composite disc 22 is configured to operate at a mode that substantially matches the operating frequency of the ultrasonic transducer 20. In some embodiments, the operating frequency of the ultrasonic transducer 20 is 1.5 MHz, and the low-volume fraction piezoelectric composite disc 22 is configured to operate in a half-wave resonant mode at 1.5 MHz.

In some embodiments, the low-volume fraction piezoelectric composite disc 22 has an acoustic impedance included in a range extending from about 9 MR to about 13 MR. In some embodiments, the acoustic impedance is about 11 MR. It should be noted that the low-volume fraction piezoelectric composite disc 22 may include any materials or combinations of materials that allows reaching the listed acoustic impedance. For example, and without being limitative, in some embodiments, the low-volume fraction piezoelectric composite disc 22 may include about 35% of PZT 5H and about 65% of a polymer matrix. The polymer matrix may include epoxy filled with micro glass balloons and silicone particles. In some embodiments, the PZT 5H has a first longitudinal acoustic velocity and the polymer matrix has a second longitudinal acoustic velocity, and the second longitudinal velocity is equal to approximately 60% to 70% of the first longitudinal velocity. For example, and without being limitative, the first longitudinal acoustic velocity may be about 3850 m/s and the second longitudinal acoustic velocity may be about 2515 m/s.

In some embodiments, the low-volume fraction piezoelectric composite disc 22 has a thickness of about λ/2 at the operating frequency of the ultrasonic transducer. For example, and without being limitative, the thickness of the low-volume fraction piezoelectric composite disc 22 may be about λ/2 at about 1.5 MHz.

As illustrated in the Figures, the ring-shaped printed circuit board 24 is in electrical contact with the low-volume fraction piezoelectric composite disc 22.

In some embodiments, the ring-shaped printed circuit board 24 has an inner diameter, and the low-volume fraction piezoelectric composite disc 22 has an outer diameter. As illustrated in the Figures, the inner diameter of the ring-shaped printed circuit board 24 may be larger than the outside diameter of the low-volume fraction piezoelectric composite disc 22. In some embodiments, the ultrasonic transducer 20 further includes a housing 30. The housing 30 may be made from plastic. In some embodiments, the housing 30 may include a matching layer in acoustic communication with the low-volume fraction piezoelectric composite disc 22. The matching layer may be integrally formed with the housing or may alternatively be provided as a separate component. In some embodiments, the matching layer may have a thickness of about λ/4. In some embodiments, the matching layer may have an acoustic impedance included in a range extending from about 2.1 MR to about 2.5 MR. In some embodiments, the acoustic impedance may be about 2.3 MR.

In some embodiments, the ultrasonic transducer 20 further includes a 2λ/3 acoustic layer in acoustic communication with the low-volume fraction piezoelectric composite disc 22. In some embodiments, the 2λ/3 acoustic layer may be made from nonyl plastic. Of note, other materials could be used. In some embodiments, the 2λ/3 acoustic layer may have a thickness of about 953 μm.

In some embodiments, the ultrasonic transducer 20 further includes a 0.9λ acoustic layer in acoustic communication with the low-volume fraction piezoelectric composite disc 22. In some embodiments, the 0.9λ acoustic layer may be made from nonyl plastic. Of note, other materials could be used. In some embodiments, the 0.9λ acoustic layer may have a thickness of about 1.28 mm.

In some embodiments, the ring-shaped printed circuit board 24 includes two opposed planar surfaces, each planar surface being made from copper.

In some embodiments, the ring-shaped printed circuit board 24 is bonded with a perimeter of the low-volume fraction piezoelectric composite disc 22.

In some embodiments, there is provided an inductor connected in parallel with a piezocomposite of the low-volume fraction piezoelectric composite disc 22, the inductor being configured to resonate electrically with the acoustic stack comprising a low-volume fraction piezoelectric composite disc 22 and the annular apodizing backing structure 26 and the ¼ lambda matching layer, such that an electrical impedance maximum is produced at about 1.5 MHz when the distal face of the ultrasonic transducer 20 is air loaded.

In some other embodiments, the inductor may be configured in series with the low-volume fraction piezoelectric composite to provide an impedance minimum instead of a maximum, as illustrated in FIGS. 19A,B, which will be presented in greater detail below.

The annular apodizing backing structure 26 is in acoustic contact with the low-volume fraction piezoelectric composite disc 22. The annular apodizing backing structure 26 includes an inner perimeter 32 having a corresponding inner thickness 34 and an outer perimeter 36 having a corresponding outer thickness 38. The annular apodizing backing structure 26 includes an inclined surface 40 forming a substantially continuous slope extending from the inner perimeter 32 to the outer perimeter 36. The inner thickness 34 is smaller than the outer thickness 28.

It should be noted that the apodizing backing structure 26 may have any shapes or configurations that allow shaping an acoustic field to reach a predetermined target which may be dictated by a targeted application. For example, and without being limitative, the apodizing backing structure 26 may have a surface profile including non-monotonic curve(s), non-continuous curve(s), or even discontinuous step(s). The geometry of the backing structure 26 depends on the apodizing needs, i.e., the optimal shape or profile of the acoustic field or the optimal transition in profile of the acoustic field. It should be noted that the present techniques that rely on attenuating an acoustic field or portions thereof using destructive interference are generally flexible and could be used to enhance the acoustic properties of a broad variety of ultrasonic transducers. For example, and without being limitative, the techniques being herein described may be used with kerf-less array, or to shape the directivity of an acoustic field generated by an annular array element.

In some embodiments, the annular apodizing backing structure 26 may include Epotek 301 epoxy.

In some embodiments, the annular apodizing backing structure 26 may have an acoustic impedance of about 2.8 MR. The annular apodizing backing structure 26 may include any materials or combinations of materials allowing to reach this acoustic impedance.

In some embodiments, the slope extending from the inner perimeter 32 to the outer perimeter 36 may be included in a range extending between about 0 degree and about 30 degrees. In some embodiments, the slope may be about 14 degrees with respect to a top surface of the low-volume fraction piezoelectric composite disc 22.

In some embodiments, the ultrasonic transducer 20 is operable at a frequency of about 1.5 MHz. In some embodiments, the ultrasonic transducer 20 may be operated in a narrow bandwidth tone burst mode, such as, for example and without being limitative, a 20% duty cycle sinusoidal pulsed mode, having a pulse repetition frequency, for example 1 kHz PRF.

Now turning to some figures of merit of the ultrasonic transducer, the present techniques provide an ultrasonic transducer 20 having a beam non-uniformity ratio of less than 3.5, which is below 8, i.e., the level being defined as a minimum safe level for physiotherapy and other medical uses. In some embodiments, the substantially uniform near field (or the at least one substantially uniform near field component or portion) exhibits less than 2 dB of ripples in a plane located at about 3 mm of an external surface of the ultrasonic transducer 20, when the ultrasonic transducer 20 is operated at 1.5 MHz with a 20% pulsed transmit waveform.

In accordance with another broad aspect, there is provided a LIPUS treatment head having an operating frequency. The LIPUS treatment head includes an acoustic stack, at least one electrode (which may be replaced or embodied by a printed circuit board and a housing). These components may be similar to one or more embodiments being herein described. The acoustic stack includes a piezoelectric disc including a low-volume fraction piezoelectric composite disc. The low-volume fraction piezoelectric composite disc is configured to operate in a half-wave resonant mode at the operating frequency of the LIPUS treatment head. The acoustic stack also includes an annular apodizing backing structure in acoustic communication with the low-volume fraction piezoelectric composite disc. The annular apodizing backing structure has an inner perimeter and an outer perimeter, respectively having an inner thickness and an outer thickness. The inner thickness is smaller than the outer thickness. The annular apodizing backing structure is configured to change an apparent thickness of the low-volume fraction piezoelectric composite disc with respect to the resonant properties of the low-volume fraction piezoelectric composite disc, thereby allowing the LIPUS treatment head to generate a substantially uniform near field (or an acoustic field including at least one substantially uniform near field component or portion). The printed circuit board in electrical communication with the low-volume fraction piezoelectric composite disc, and the housing is shaped and sized for supporting the acoustic stack and the printed circuit board.

Of note, the LIPUS treatment head and each of its components are compatible with the embodiments having been previously described with respect to the ultrasonic transducer.

In accordance with another broad aspect, there is provided an apodizing wedge structure for a LIPUS treatment head, the LIPUS treatment head including a low-volume fraction piezoelectric composite disc. The apodizing wedge structure includes an annular body for contacting a surface of the low-volume fraction piezoelectric composite disc. The annular body includes an inner perimeter having a corresponding inner thickness and an outer perimeter having a corresponding outer thickness. The annular body includes an inclined surface forming a substantially continuous slope extending from the inner perimeter to the outer perimeter, the inner thickness being smaller than the outer thickness. The apodizing wedge is configured to change an apparent thickness of the low-volume fraction piezoelectric composite disc with respect to resonant properties of the low-volume fraction piezoelectric composite disc when the apodizing wedge structure is in acoustic communication with the low-volume fraction piezoelectric composite disc, thereby allowing the LIPUS treatment head to generate a substantially uniform near field (or an acoustic field including at least one substantially uniform near field component or portion).

In some embodiments, the annular body is made from Epotek 301 epoxy.

In some embodiments, the annular body has an acoustic impedance of about 2.8 MR.

In some embodiments, the slope is comprised between 0 degrees and 30 degrees. In some embodiments, the slope is about 14 degrees with respect to a top surface of the low-volume fraction piezoelectric composite disc.

In some embodiments, the LIPUS treatment head is operable at a frequency of about 1.5 MHz.

In some embodiments, the LIPUS treatment head is operable in a narrow bandwidth tone burst mode. In some embodiments, the narrow bandwidth tone burst mode is a 20% duty cycle sinusoidal pulsed mode.

In some embodiments, the LIPUS treatment head has a beam non-uniformity ratio of less than 3.5.

In some embodiments, the substantially uniform near field (or the at least one substantially near field component or portion) exhibits less than 2 dB of ripples in a plane located at about 3 mm of an external surface of the LIPUS treatment head, when the LIPUS treatment head is operated at 1.5 MHz with a 20% pulsed transmit waveform.

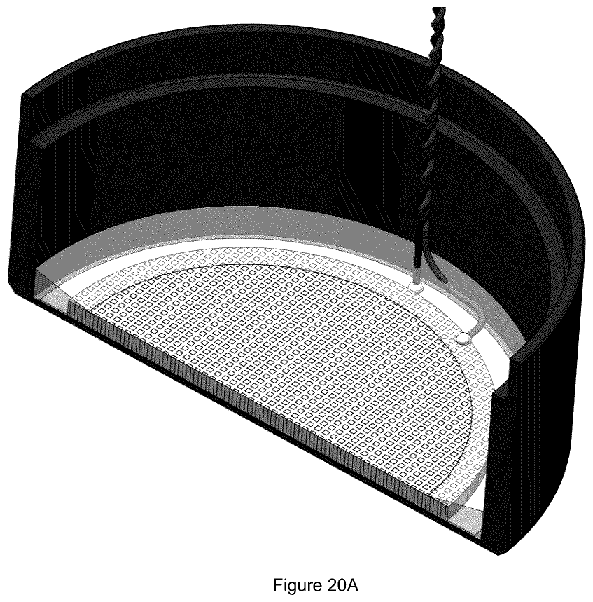

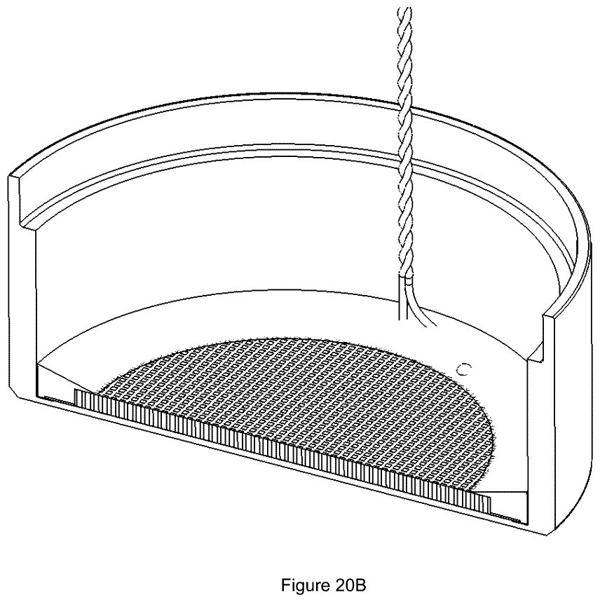

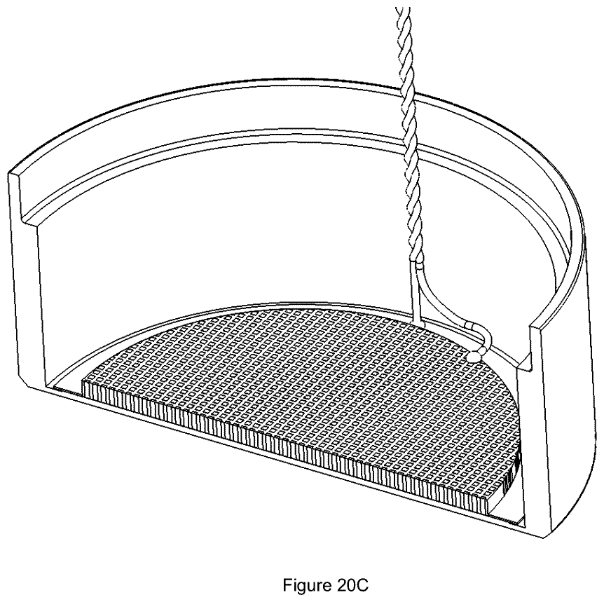

FIGS. 20A-C show an ultrasonic transducer, in accordance with another embodiment. Of note, the ultrasonic transducer of FIGS. 20A-C do not include a ring-shaped printed circuit, as described elsewhere. The ultrasonic transducer according to this embodiment may however include the low-volume fraction piezoelectric composite, the at least one electrode and the annular apodizing backing structure as herein presented. In the embodiment illustrated in FIGS. 20A-C, the at least one electrode is represented as a ring at least partially surrounding the low-volume fraction piezoelectric composite. The electrode may be made from a metallic material, such as gold, for example.

The technology having been insofar described may be described in terms of a nonlimitative embodiment of a LIPUS treatment head that will now be presented. In this nonlimitative embodiment, the LIPUS treatment head is configured to operate at a frequency of approximately 1.5 MHz. Such a LIPUS treatment head may be used in a fracture healing therapy system or similar systems. The LIPUS treatment head according to this embodiment may be used to reduce or optimize the time required to heal bone fractures, assist or promote healing of relatively complex or difficult open fractures, and in physiotherapy diathermy systems.

Broadly described, the LIPUS treatment head according to this embodiment includes an ultrasonic transducer and integral gel sensing electrical impedance function. More specifically, the LIPUS treatment head may include a low volume fraction 1 3 piezoelectric composite disc, a ring-shaped printed circuit board, a ground electrode, a signal electrode, a twisted pair of wires, a plastic housing, an apodizing wedge and a printed circuit board.

The low volume fraction 1 3 piezoelectric composite disc may have a diameter included in the range extending between 23 mm and 24 mm, and preferably about 23.6 mm diameter. The low volume fraction 1 3 piezoelectric composite disc may have a thickness of about ½ lambda at 1.5 MHz. or about 960 μm in the longitudinal axis. The low volume fraction 1 3 piezoelectric composite disc may have may include 280 μm square pillars forming a 2D matrix pattern. The 2D matrix may have a pitch of about 480 μm in both lateral axes. The low volume fraction 1 3 piezoelectric piezocomposite disc may be configured to operate in a half-wave resonant mode at about 1.5 MHz. The piezoelectric composite disc may exhibit acoustic impedance included. for example and without being limitative, in a range extending from about 9 MR to about 13 MR. and preferably about 11 MR. The piezoelectric composite disc may include., by volume, approximately 35% of PZT 5H pillars, and approximately 65% (i.e., a remaining portion) of polymer matrix. The piezoelectric composite disc may be manufactured according to a dice and fill method. The polymer matrix may include, for example and without being limitative, an approximately 2.2 MR powder loaded Epotek 301 epoxy filled with micro glass balloons and silicone particles. The polymer matric may have a longitudinal acoustic velocity equal to approximately 2515 m/s, or approximately 60% to 70% of the longitudinal bar mode velocity of the PZT pillars. In some embodiments the longitudinal bar mode velocity of the PZT pillars may be about 3850 m/s.

The ring-shaped printed circuit may act as a perimeter support to the piezoelectric composite disc, may be referred to as a perimeter support PCB (PSPCB). The PSPCB generally has an inner diameter that is slightly larger than the outside diameter of the piezoelectric composite disc, and an outer diameter that is designed to accommodate the inner diameter of the LIPUS treatment housing. The PCB may have copper conductive planes on distal and proximal faces, covering most of a respective face, and the proximal electrode may be separated into two regions, each electrically isolated one from another. One of the two regions may contain a via creating electrical communication between the distal copper plane and the section of the proximal plane containing the via. It will be understood that the PSPCB generally includes a plurality of different layers, and so may be provided in many different configurations. The PSPCB may be bonded with, for example and without being limitative, epoxy, to the perimeter of the piezoelectric composite disc, such that the distal face of the piezoelectric composite disc extends slightly past the distal face of the PSPCB, for example and without being limitative by approximately 50 μm to approximately 100 μm.

The electrically conductive electrodes may be provided according to methods and techniques known in the art. A nonlimitative example of the electrically conductive electrodes are chrome-gold electrodes. and they may be provided on the proximal and distal surfaces of the piezoelectric composite disc. In some embodiments, the electrically conductive electrodes may be formed using a deposition technique, such as, for example and without being limitative, sputtering. Of note, the electrically conductive electrode provided on the distal surface (sometimes referred to as a “distal electrode”) of the piezoelectric composite disc may act as a ground electrode for the LIPUS treatment head. The distal electrode may establish electrical communication between the distal surface of the piezoelectric composite disc and the distal copper plane of the PSPCB. The electrically conductive electrode provided on the proximal surface (sometimes referred to as a “proximal electrode” may act as a signal electrode of the LIPUS treatment head. The proximal electrode may establish electrical communication between the proximal surface of the piezoelectric composite disc and the isolated proximal copper plane of the PSPCB.

The pair of twisted wires is electrically connected to the proximal surface of the PSPCB, and may be, for example and without being limitative. soldered to the proximal surface of the PSPCB. A first one of the pair of twisted wires may be a ground wire and be configured to make electrical contact with the distal electrode (i.e., the ground electrode) by way of the portion of the proximal copper plane containing the via in the PSPCB. A second one of the pair of twisted wires may be a wire and be configured to make contact with the proximal piezoelectric composite electrode by way of the isolated proximal copper plane of the PSPCB.

The plastic housing may include an integral single quarter-wavelength thickness matching layer. The matching layer has an acoustic impedance in the range of about 2.1 MR to about 2.5 MR, and preferably about 2.3 MR. The matching layer may be made, for example and without being limitative, from HNA055 Noryl PPO plastic. The matching layer may have a thickness of about 360 μm. The matching layer is in acoustic communication with the front surface (i.e., the distal surface) of the piezoelectric composite disc, and may be configured to perform optimally in a continuous wave or narrowband tone burst mode. The acoustic impedance of materials may become significantly lower in continuous wave resonant conditions, and quarter wave matching layers can be optimized for varying acoustic applications.

The apodizing wedge is in acoustic communication with a portion of the back surface (i.e., the proximal surface) of the piezoelectric composite disc, wherein the proximal electrode is interposed therebetween. The apodizing wedge has an acoustic impedance that is comparable to or somewhat higher than the acoustic impedance of the matrix material of the piezoelectric composite. The apodizing wedge may include, for example, and without being limitative, Epotek 301 epoxy. The apodizing wedge may have an acoustic impedance of approximately 2.8 MR. The apodizing wedge acts to change the apparent thickness of the piezoelectric composite disc with respect to the resonant properties of the piezoelectric composite disc in the location of communication between the piezoelectric composite disc and the apodizing wedge. More specifically the apodizing wedge adds to the acoustic path length of the piezoelectric composite disc and produces strongly destructive 180 out-of-phase reflections from the proximal surface of the apodizing wedge when it is λ/4 thickness. In some embodiments, the λ/4 thickness may be about 440 μm. The apodizing wedge is radially tapered in thickness with respect to the radial dimension of the piezoelectric composite disc, such that the wedge is λ/4 thick at the perimeter of the piezoelectric composite disc and tapering down to zero thickness for example near the inner diameter. The apodizing wedge may be radially taped with an angle of about 14 degrees with respect to the proximal surface of the piezoelectric composite disc. The apodizing wedge and the piezoelectric composite act together to effectively produce a monotonically decreasing level of destructive interferences as the thickness monotonically tapers down to zero, at which point the piezoelectric composite disc experiences the usual λ/2 fully constructive interference between the front and back wall reflections of the low-volume fraction piezoelectric composite disc and the apodizing backing structure.

Of note, a volume of air is in contact with the proximal surface of the apodizing wedge and the exposed portion of the proximal surface of the piezoelectric composite disc. As such, the proximal surfaces of the transducer in the LIPUS treatment head according to this nonlimitative embodiments is acoustically loaded with air.

The LIPUS treatment head may include a series or parallel connected inductor, for example, a 2 μH inductor, connected in parallel with the piezoelectric composite disc of the LIPUS treatment head. The inductor may be configured to resonate electrically with the acoustic stack, when it is air loaded, such that an impedance maximum is produced at the operating frequency of the LIPUS treatment head for a parallel inductor or an impedance minimum is created for a series resonant inductor. The operating frequency of the LIPUS treatment head may be 1.5 MHz, the distal surface (or at least a portion of the distal surface) of the LIPUS treatment head is acoustically loaded with air.

The ultrasonic transducer according to this nonlimitative embodiment is configured to produce a highly uniform acoustic field, which one skilled in the art will appreciate can be beneficial in many applications, and particularly in medical LIPUS applications such as physiotherapy transducers and fracture healing applications when low energy is to be applied to a patient without image guidance. One example of a fracture healing system may be configured to operate at a center frequency of about 1.5 MHz in a narrow bandwidth tone burst mode. A nonlimitative example of the narrow bandwidth tone burst mode is a 20% duty cycle sinusoidal pulsed mode having a PRF of about 1 kHz.

One skilled in the art will be familiar with the beam non-uniformity ratio (RBN), which is for example and without being limitative, described in IEC61689:2013, as the ratio of the spatial peak temporal average intensity (ISPTA) of the acoustic field of the transducer divided by the spatial average temporal average (ISATA) intensity of the transducer measured at a plane orthogonal to the axis of the beam, located at a distance of 3 mm from the face of the ultrasonic transducer. R_{BN} is a figure of merit for the safety and efficacy of physiotherapy transducers and medical LIPUS transducers in many applications, and that an RBN of less than 8 is defined as a minimum safe level for physiotherapy and other medical uses. Of note, the actual industry average is typically less than 6, and the median is typically about 3.7. The present technology is capable of producing an R_{BN} of less than 3.5, with an extremely uniform near field exhibiting less than 2 dB of ripple in the 3 mm plane of the near field when operated at 1.5 MHz with a 20% pulsed transmit waveform. In addition to a uniform R_{BN}, the nonlimitative embodiment of the LIPUS treatment head being described exhibits a highly uniform axial response in the near field, which may be a desirable quality for patient comfort and uniform treatment of the patient.

In addition, due to the combination the air-backed low-volume fraction piezoelectric composite disc and the destructive-interference based apodization wedge, the LIPUS treatment head according to this nonlimitative embodiment is very efficient, having no absorbing structures in the acoustic stack. The lack of lossy absorbing structures, combined with an efficient low-volume fraction piezoelectric composite disc exhibiting typical k_{eff} values (e.g., k_{eff}>0.6 with ordinary PZT5H), allows the present technology to provide an efficiency advantage over existing solutions that generally rely on absorbing backing structures and attenuative filters on the front of the ultrasonic transducers to produce low RBN acoustic fields. The present technology can therefore potentially enable extended battery life and may allow cost effective electronics to be used to drive it in many typical physiotherapy and other medical applications.

In addition to efficient operation, a significant advantage of the technology is the realization of an efficient acoustic design that produces a uniform apodized acoustic field and does so using an air backing. Using an undamped air backing design allows the impedance sensing of the acoustic load on the front of the LIPUS treatment head to be highly efficient. One skilled in the art will know that state-of-the-art LIPUS treatment heads (and their inherent transducers) typically exhibit impedance changes due to the distal face of the treatment head being either air or gel/water coupled, and that these impedance changes are generally observed to be higher impedance in the water coupled condition compared to the air coupled condition and often by more than two times higher impedance. This impedance change leads to the transducer typically exhibiting higher electrical current flow when air coupled, or imperfectly water coupled, compared to when water or tissue coupled.

The inherent impedance change exhibited by the present techniques may be used to enable a gel sensing function by, for example, measure the average electrical current flowing through the transmit circuit connected to the LIPUS treatment head. The difference in impedance when water is coupled compared to when air is coupled makes gel sensing simple for the system designer, and allows for potentially greater discrimination between ideal coupling, partial acoustic coupling with the patients tissue, or no acoustic coupling, as in a case for example, when a patient may have forgotten to use a typical acoustic couplant gel, thus allowing the system to signal an error state with potentially great feedback to the patient or medical practitioner. Of note, some properties or figures of merits (e.g., high Q, high impedance resonant maxima) cannot be achieved in this manner on a conventional ultrasonic transducer that includes an acoustically damping backing structure, or otherwise highly damped acoustic structure.

The magnitude of the impedance when air or water is coupled varies by less than 10% when an optimal acoustic matching layer is employed on the distal surface of the piezoelectric composite disc, when no resonating inductor is included in the LIPUS treatment head. This is generally considered to be too little impedance difference to be of use for a medical or physiotherapy gel sensing function. In the case of the present technology however, due to the highly undamped and therefore highly resonant state of the air backed piezoelectric composite disc, and in conjunction with the use of an efficient λ/4 matching layer in acoustic communication with the distal surface of the piezoelectric composite disc, one preferred embodiment of the present technology incorporates the use a parallel inductor, for example, 2 μH, selected to resonate with the capacitive reactance of the piezoelectric composite disc when air is loaded on the distal face, in conjunction with the use of a λ/4 matching layer. The resulting LC resonance produces a high-Q impedance maximum at the desired operating frequency when the distal face of the λ/4 matching layer is air loaded. When the transducer is placed in contact with gel or tissue, or another similar acoustically conductive medium, the resonant maxima is diminished as the transducer is increasingly coupled to the gel or tissue, resulting in a lower impedance at the operating frequency until it is no longer electrically resonant with the water or gel loaded impedance of the piezocomposite. This approach can result in an impedance maxima magnitude of between 80 to 100 ohms and approximately +40 degrees phase at 1.5 MHz when air loaded. compared to 18 to 24 ohms at approximately +36 degrees phase when coupled to water, or a difference of, for example, between 3 to 5 times the impedance magnitude exhibited when the distal surface face of the LIPUS treatment head is in contact with water, gel, or tissue (i.e., when the LIPUS treatment head is coupled to the patient). Furthermore, the effect of the parallel inductor becomes innocuous to the functioning of the transducer when it is coupled to the patient, resulting in less than 1 dB output drop when driven by a suitable low output impedance transmit circuit, for example and without being limitative, a transmit circuit having <10 ohms output impedance, making possible an inbuilt impedance-based gel sensing function in the transducer that is several times more sensitive than relying on the inherent impedance changes of the acoustic structure of the transducer alone. In addition, this method decouples the thickness and other acoustic properties of the layer or layers on the front or distal face of the LTH, from the function of producing a gel sensing impedance change, allowing designers to optimise both the gel sense impedance sensitivity and the layer or layers on the front of the transducer for maximum acoustic efficiency, enhanced bandwidth or impulse response or other desirable acoustic properties.

One skilled in the art would have readily understood that that the present technology can be paired with many different configurations of acoustic and protective front layers in acoustic communication with the distal surface of the piezoelectric composite disc, such that a variety of impedance changes are possible without limiting or compromising the acoustic uniformity provided by the combination of the disclosed low-volume fraction piezoelectric composite disc and the apodizing wedge.

In one variant of this nonlimitative embodiment of the LIPUS treatment head, the LIPUS treatment head includes a 0.66λ acoustic layer. The 0.66λ may be a 953 μm thick nonyl plastic layer. The 0.66λ is in acoustic communication with the distal surface of the piezoelectric composite disc (instead of a λ/4 layer), which results in an impedance maximum occurring at the operating frequency of 1.5 MHz, when air is loaded on the distal surface, having magnitude of 3 to 5 times more than the water coupled condition. Of note, the thickness tolerance of the 0.66λ may be managed during, for example, manufacturing processes, in order to maintain the impedance change required for sensing the presence of adequate gel or tissue coupling to the patient. It should be further noted that the present technology in this configuration results in a highly uniform beam having R_{BN} of less than 3.5.

In another variant of this nonlimitative embodiment of the LIPUS treatment head, the LIPUS treatment head may include a 0.9λ acoustic layer. The 0.9λ acoustic layer may be a 1.28 mm thick noryl to achieve an impedance minimum at the operating frequency of 1.5 MHz when air is loaded on the distal surface of the layer, achieving a difference of approximately 3 times that of the water coupled condition. This embodiment can also be configured in conjunction with the present technology to produce an acoustic field having an R_{BN} of less than 3.5.

It should be noted, however, that any layer other than an acoustically matching layer (e.g., a ¼ lambda), or an odd multiple of ¼ lambda (e.g., ¾ lambda or 5/4 lambda), will limit the bandwidth and potentially the efficiency of the ultrasonic transducer. In light of this consideration, it may be sometimes advantageous to utilize one aspect of the present technology, being the inclusion of a resonating inductor, to enable large impedance changes, sensitive to acoustic loads present at the distal face of the LIPUS treatment head, in a manner that does not limit the use of, for example, a ¼ wave matching layer. While generally low bandwidth applications such as LIPUS applications do not strictly require transducers having a short, broadband impulse response, there remain aspects of the ultrasonic transducers performance that benefit from a broader bandwidth, such as the ability to quickly attain the steady state pulse intensity, and to limit ringdown after the transmit pulse has ended.

Of note, numerous methods for preparing low-volume fraction piezoelectric composite disc (or similar structures) are known in the art. Nonlimitative example of such methods include dice and fill methods, that would be equally applicable to the present technology. In some embodiments, the apodization techniques having been herein described can be applied to many different geometries, to achieve apodization or beam shaping of the output of a transducer of a wide possible array of beam shapes. The apodization techniques may be applied to ID arrays, 2D matrix arrays, annular arrays, single element broadband transducers and material specific carbon fiber transducers for NDT and many other applications.

In accordance with another aspect, there is provided a method of apodizing an acoustic field. The method includes operating an ultrasonic transducer to generate an acoustic field, the ultrasonic transducer being similar to one or more of the embodiments being herein described. The method also includes conditioning the acoustic field with an annular apodizing backing structure to generate an apodized acoustic field, the apodized acoustic field in at least one substantially uniform near field component or portion, the annular apodizing backing structure being in acoustic contact with the low-volume fraction piezoelectric composite disc. The annular apodizing backing structure is similar to one or more of the embodiments being herein described.

In some embodiments the ultrasonic transducer is operated at 1.5 MHz with a 20% pulsed transmit waveform, and the substantially uniform near field exhibits less than 2 dB of ripples in a plane located at about 3 mm of an external surface of the ultrasonic transducer.

In some embodiments, the low-volume fraction piezoelectric composite disc is in a 1 3 configuration.

In some embodiments, the low-volume fraction piezoelectric composite disc includes 280 μm by 280 μm pillars distributed in a 2D matrix pattern having a pitch of about 480 μm in both lateral axes.

In accordance with another aspect, there is provided a method for generating an acoustic field with a low-intensity pulsed ultrasound (LIPUS) treatment head having an operating frequency. The method includes operating the LIPUS treatment to generate the acoustic field, the LIPUS treatment head being similar to one or more of the embodiments being herein described. The method also includes conditioning the acoustic field with the annular apodizing backing structure to generate an apodized acoustic field, the apodized acoustic field having a substantially uniform near field region.

In some embodiments the ultrasonic transducer is operated at 1.5 MHz with a 20% pulsed transmit waveform, and the substantially uniform near field exhibits less than 2 dB of ripples in a plane located at about 3 mm of an external surface of the ultrasonic transducer.

In some embodiments, the low-volume fraction piezoelectric composite disc is in a 1 3 configuration.

In some embodiments, the low-volume fraction piezoelectric composite disc includes 280 μm by 280 μm pillars distributed in a 2D matrix pattern having a pitch of about 480 μm in both lateral axes.

### Results

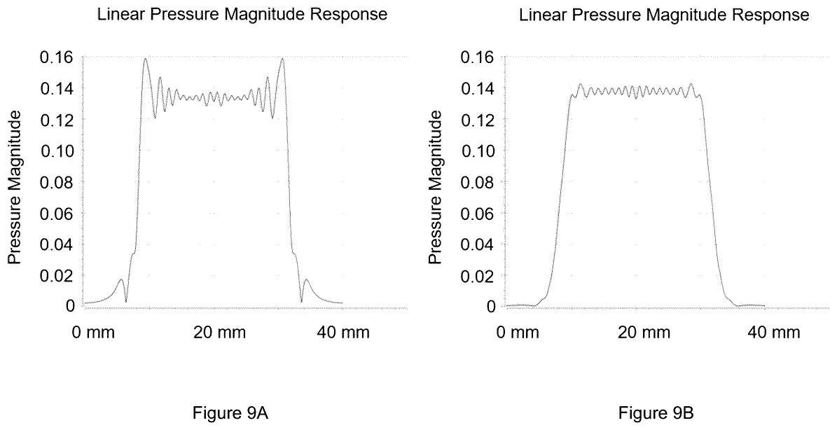

Now that different embodiments of the technology have been described, the performances of some of these embodiments will be discussed, and more specifically in terms of the results that may be obtained using the present techniques. Nonlimitative examples of results are illustrated in FIGS. 9A,B to 19A,B.

In FIGS. 9A,B, there are illustrated a linear pressure magnitude response of a conventional ultrasonic transducer (left portion) and a linear pressure magnitude response of an ultrasonic transducer designed according to the present techniques (right portion). The results illustrate the 2D lateral acoustic pressure at a 3 mm plane from the face of the ultrasonic transducer. The impact of the apodization wedge (i.e., the annular apodizing backing structure) on the uniformity of acoustic field is clearly represented.

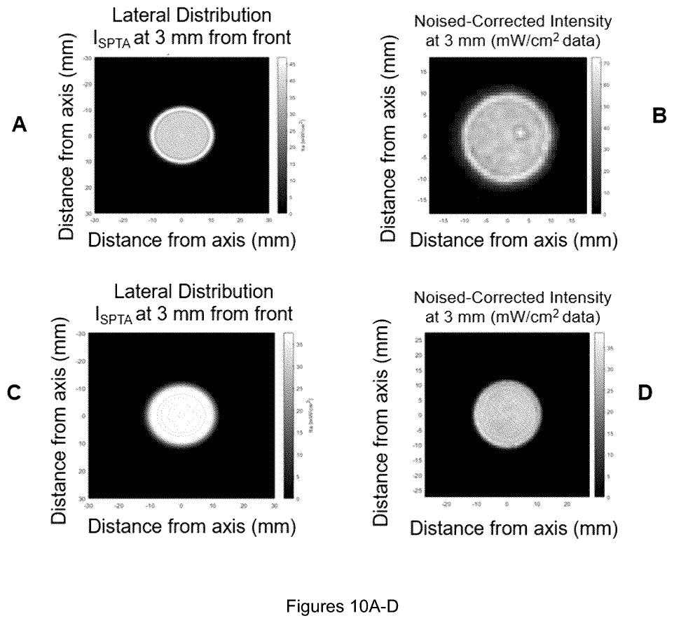

In FIGS. 10A-D, there are illustrated a simulated field showing outer maximum ring present in a 3 mm field of a 1 3 composite-based LIPUS treatment head with no apodization ring or structure (top left portion): a measured acoustic field showing outer maximum ring present in the 3 mm field of the 1 3 composite-based LIPUS treatment head (top right portion): a simulated field showing suppressed outer maximum ring resulting from the inclusion of an apodizing wedge (or an “annular apodizing backing structure) on back perimeter of the 1 3 composite-based disc (bottom left portion); and a measured acoustic field from a prototype LIPUS treatment head showing suppressed outer maximum ring achieved with the apodizing ring (or annular apodizing backing structure) on the 1 3 composite-based disc (bottom right portion).

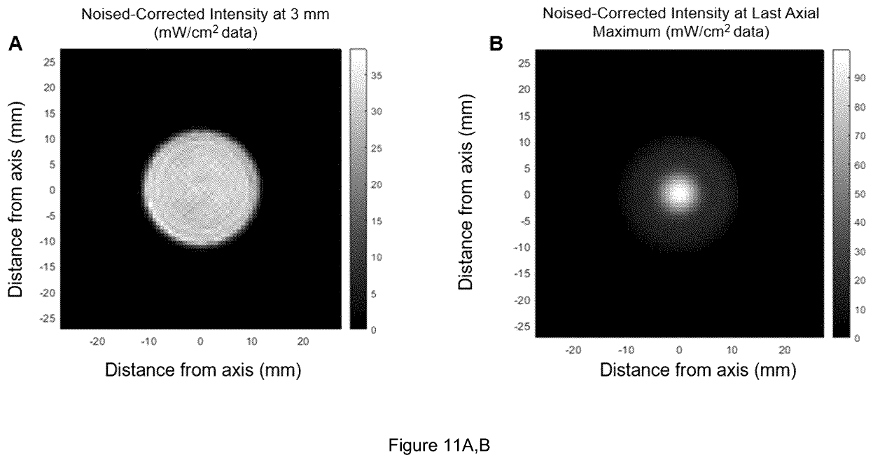

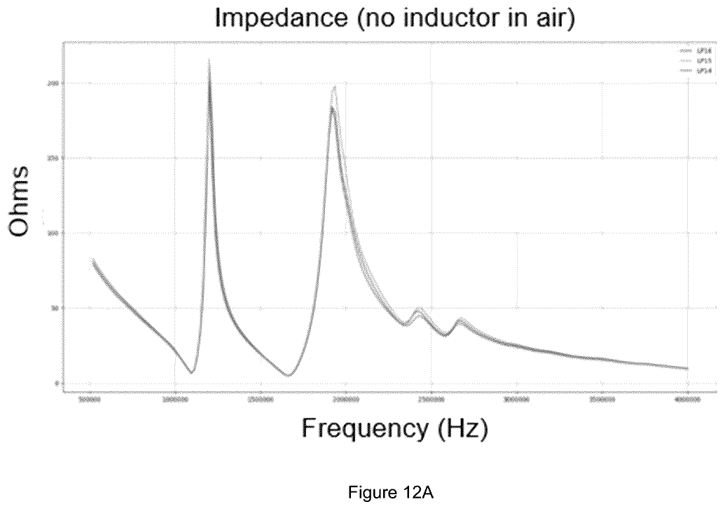

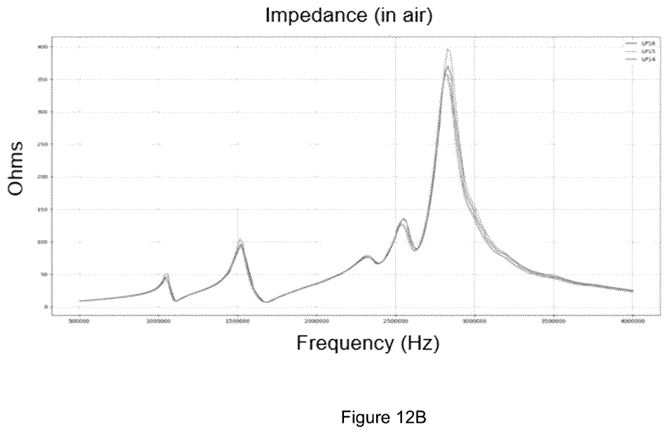

FIGS. 11A,B show the 3 mm intensity and the last axial maximum intensity of the present techniques. These results illustrate that the beam produced with the present technology has desirable properties for medical and physiotherapy applications. The figures of merit include: AER=3.9 cm², RBN=3.25, and collimated beam type at an output power of 117 mW and intensity of 30 mW/cm² measured at the plane located 3 mm away from the distal face of the LIPUS treatment head.

FIGS. 12A and 12B illustrate the effect of air loading with no inductor (FIG. 12A) compared to air loading with a parallel inductor configured to resonate at 1.5 MHz (FIG. 12B) of the present technology, with parallel inductive resonance tuned to match operating frequency when the ultrasonic transducer is air loaded.

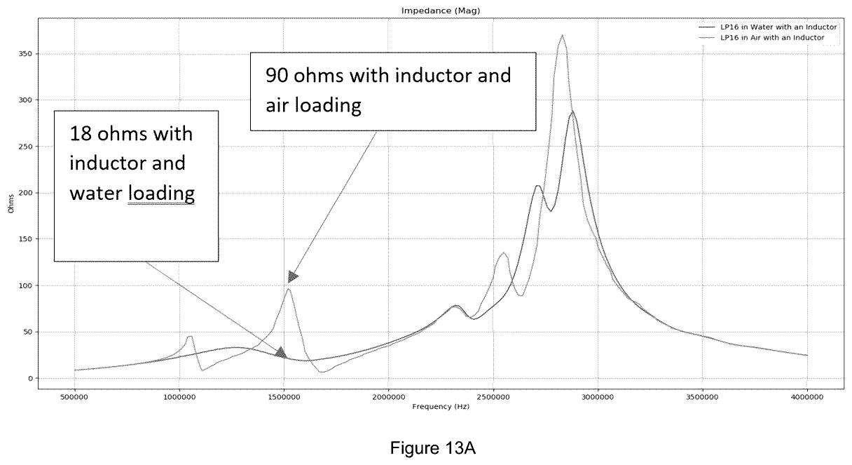

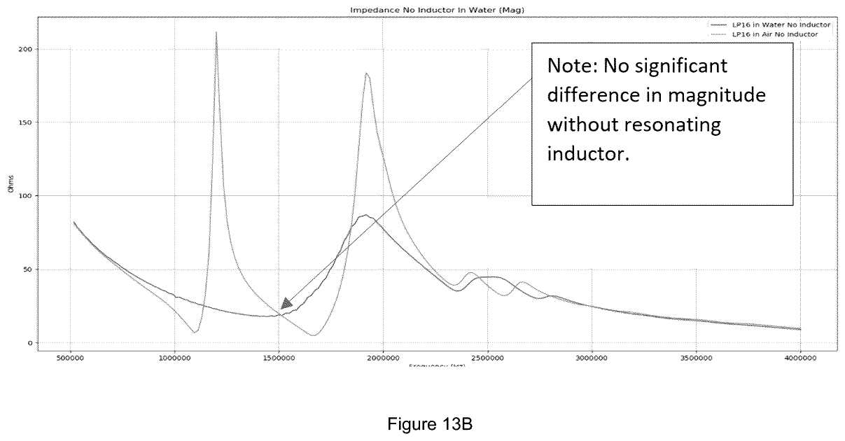

FIG. 13A shows the impedance of the nonlimitative embodiment of the LIPUS treatment head having a λ/4 matching layer and a resonating parallel inductor with air and water loading. FIG. 13B shows the impedance of the nonlimitative embodiment of the LIPUS treatment head Exemplary transducer having a λ/4 matching layer but no resonating inductor, with air and water loading cases.

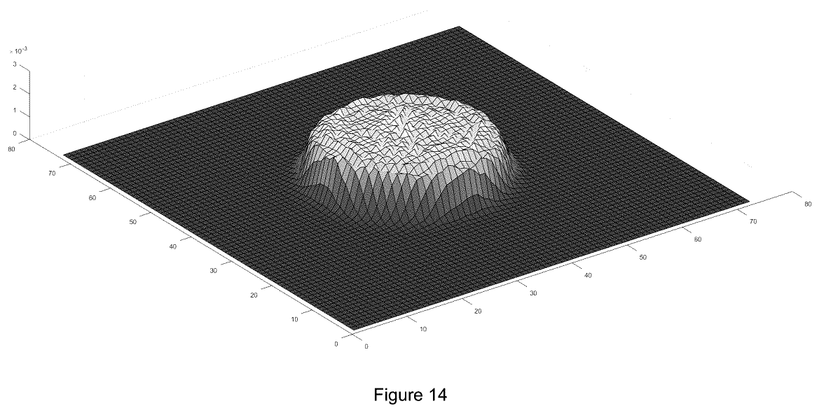

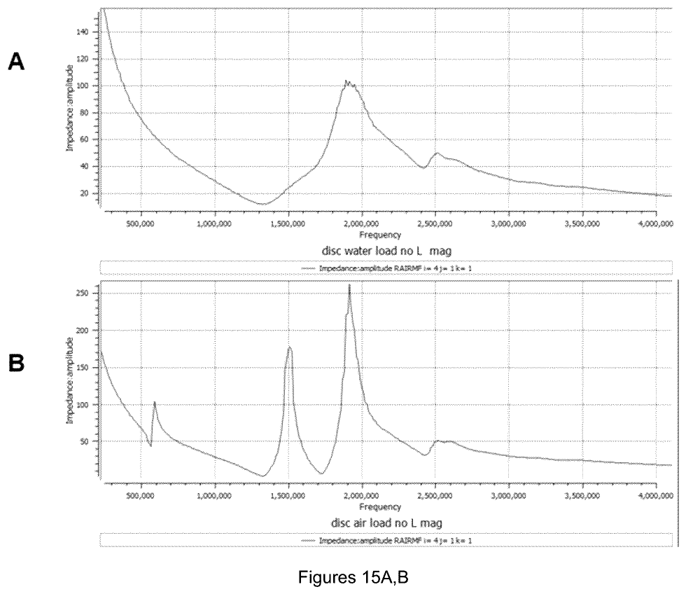

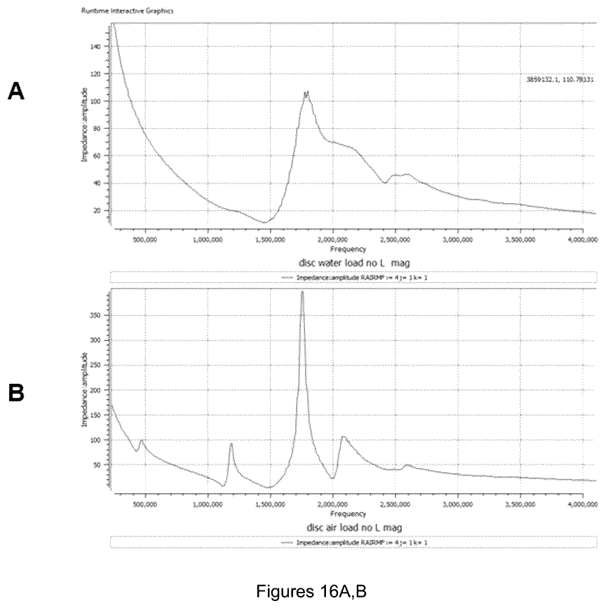

FIG. 14 is a 3D plot of an apodized acoustic field generated with the present techniques, measured in water tank with hydrophone.

FIGS. 15A,B show a thicker 2λ/3 front layer tuned to provide impedance maxima (shown in the bottom half of the figure) at operating frequency in air without the use of a resonant inductor in the circuit, compared to the response in water (shown in the top half of the figure), the air coupled maximum being approximately 5 times higher than the water coupled minimum.

FIGS. 16A,B shows a still thicker 0.9λ front layer tuned to provide an impedance minimum in air (shown in the bottom half of the figure), and a higher impedance when water coupled (shown in the top portion of the figure), the air-coupled impedance minimum being approximately 3 times lower than that when water coupled (bottom portion).

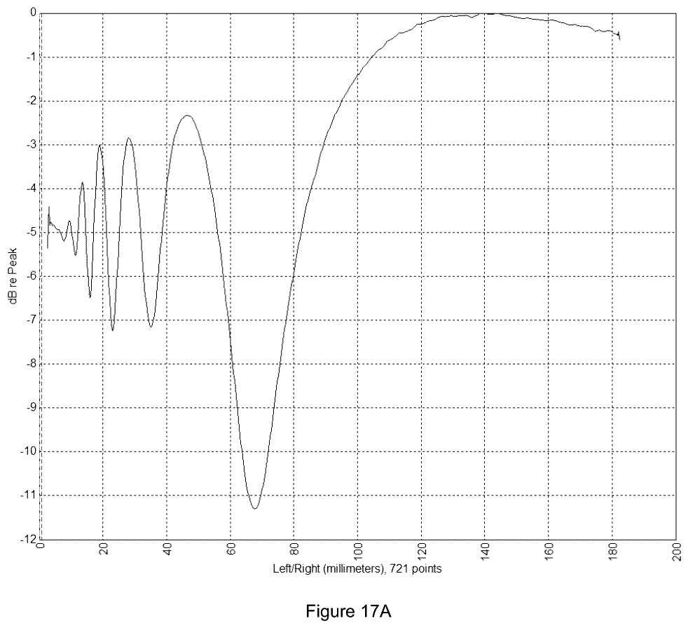

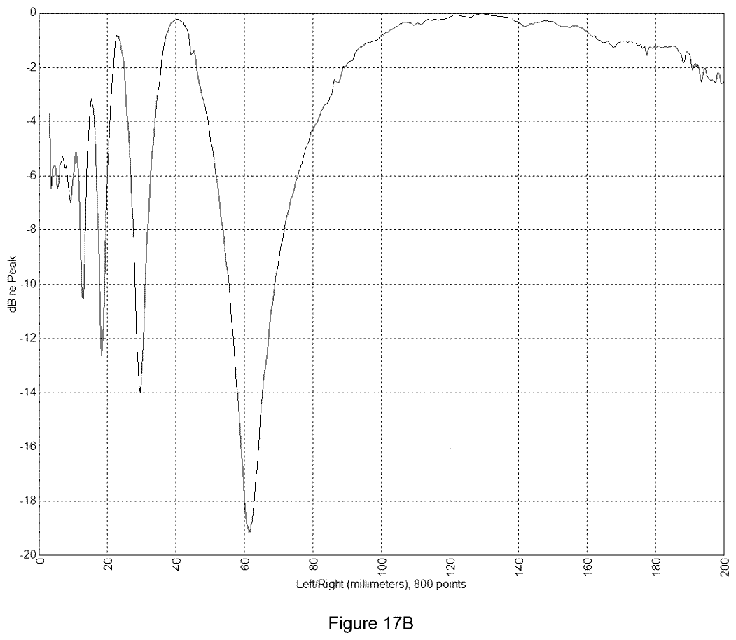

FIGS. 17A,B show the axial pressure response of the ultrasonic transducer, according to the present techniques. FIG. 17A illustrates a highly uniform on-axis pressure field in the first 10 cm of the near field, and an absence of high amplitude peaks in the entire near field. Figure B illustrates the lack of near field axial uniformity seen in the absence of the apodization backing structure and corresponding acoustic field.

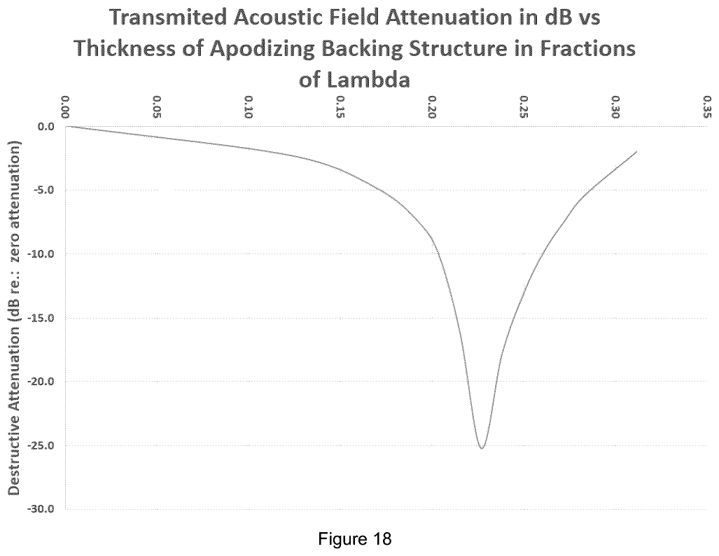

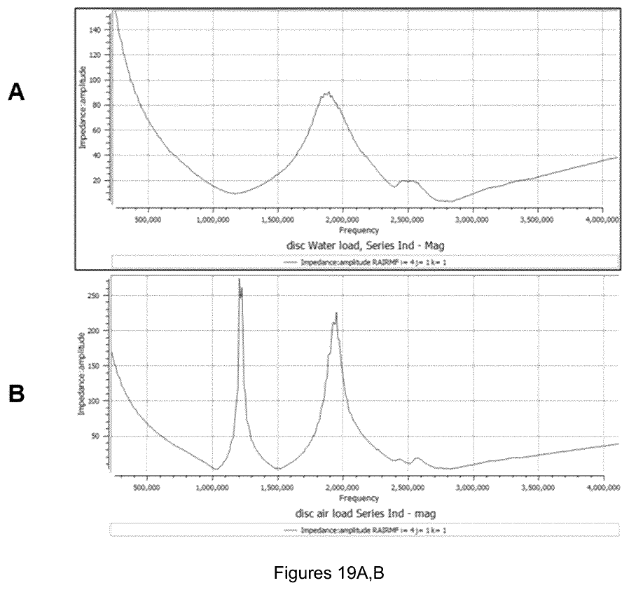

FIG. 18 illustrates the localized attenuation of a transmitted acoustic field due to destructive interference generated by the presence of the backing structure versus the thickness of the backing structure as a fraction of the wavelength. Of note, the total thickness of the backing structure and the composite kerf filling matrix should be equal to approximately ¾ lambda for maximum attenuation, and so the minimum is observed at a thickness equal to about ¼ lambda of the backing structure when the speed of sound in the matrix of the composite is slower than that of the backing structure.

FIGS. 19A,B are representative of a method of achieving the result seen in the 0.9λ case but using a series resonant inductor and λ/4 matching layer. More specifically, FIGS. 19A,B show a comparison of the impedance of a variant of the exemplary embodiment LIPUS treatment head, including a series resonant inductor and a λ/4 matching layer design configured to produce an impedance minimum when air coupled that is smaller than the impedance when water loaded, allowing gel sense circuits to detect proper coupling when current flowing through the circuit is at a lower level then when the transducer is air coupled. In this example, the air minimum is about 4 ohms, the water coupled impedance magnitude at the operating frequency is about 24 ohms (a sixfold increment), which may be useful in gel sensing applications.

It should be noted that the results having been described serve an illustrative purpose only and should therefore not be considered limitative.

### Examples

Now that different embodiments of an apodizing backing structure and its integration in ultrasonic transducers have been described, as well as results that can be obtained using the present techniques, nonlimitative examples of the technology will now be described.

In a first example, the ultrasonic transducer or the LIPUS treatment head includes a 1 3 piezocomposite disc, including a low volume fraction of piezo material (35% PZT 5H). The balance of the piezocomposite disc includes a filled epoxy matrix (65% filled epoxy matrix). The filled epoxy matrix has an acoustic impedance of about 2 MR, effectively isolating the lateral vibrations of each pillar from adjacent pillars. resulting in a near k₃₃ bar mode resonance from the PZT pillars. The piezocomposite disc has a low lateral coupling efficiency between the PZT pillars, resulting in most of the acoustic energy being generated in the axial direction of the disc. The ultrasonic transducer or the LIPUS treatment head also includes an apodizing ring having a wedge-shaped cross section, located on a portion of the back face of the piezo composite located near the perimeter of the disk, the apodizing wedge including an epoxy having acoustic impedance that closely matches that of the matrix portion of the piezocomposite. The wedge may include Epotek 301 epoxy having an acoustic impedance of about 2.8 MR. The apodizing wedge effectively changes the acoustic resonant frequency of the low volume fraction 1 3 composite disc, shifting the resonant frequency of the disc lower as the wedge becomes thicker. The wedge ultimately approaching the thickness equating to λ/4 at the driving frequency of the transducer, and thus facilitating destructive interference within the piezocomposite disc, the wedge structure creating a strongly attenuating effect that varies with the thickness of the wedge. The apodizing structure on the back face of the piezocomposite accomplishes local attenuation of the resonant behavior of the piezocomposite disc without absorbing significant acoustic energy making it behave as a different thickness piezo disc. The apodizing wedge provides strong destructive interference for narrow band acoustic signals such as a tone burst for example, and so may attenuate the acoustic output of the transducer by approximately 25 dB for a tone burst of 50 cycles for example. The shape of the apodizing structure, for example, a wedge, is able to shape the spatial apodization and may result in a gradual increase in amplitude with radial distance from the center of the disc as the phase of the interference within the apodizing wedge and disc thickness increases from a fully constructive λ/2 to a fully destructive 3λ/4 lambda. A parallel tuned inductor circuit may be provided, the inductor is designed to resonate with the air loaded impedance of the ultrasonic transducer or the LIPUS treatment head at the operating frequency of the LIPUS transducer, such that an impedance maximum is created at the operating frequency of the transducer. The matching layer of the transducer works in conjunction with the stack and parallel inductor such that the inductor does not cause a substantial resonance at the operating frequency of the LIPUS transducer when the LIPUS transducer is water loaded, gel loaded or tissue loaded.

A second example relates to acoustic field and lateral reverberations in 1 3 piezoelectric composites with an apodizing wedge in narrow bandwidth operation mode. In this second example, the ultrasonic transducer or the LIPUS treatment head includes a 1 3 piezocomposite with an apodizing wedge for reducing side lobe amplitude by applying apodization to the perimeter of the piezocomposite element. Using a low-volume fraction 1 3 piezocomposite helps in reducing lateral resonances in the circular disc, which in turn reduces the complexity of the near field interference patterns generated by the disc, making the near field uniform laterally to within less than about 2 dB. This idealized near field results in very low ripple in the 3 mm plane that is used to characterize medical physiotherapy. diathermy, and fracture healing LIPUS transducers. Low lateral resonance in the disc also reduces the on axis non-idealities that can arise from constructive and destructive interference between the main axial mode of the piezocomposite disc and axial components generated by lateral resonances in the disc. Axial uniformity is an important consideration in the efficacy and patient comfort of LIPUS-based medical devices. The ability to shape the edges of the acoustic field through the apodizing backing structure herein disclosed is significant in that beyond simply reducing side lobes. one may shape the acoustic field so that the main lobe is of an ideal shape. The apodizing backing structure can be optimized to reduce the output of the transducer by more than −25 dB for narrowband operation. while using only λ/4 thickness backing structure. In addition, it can be shaped to produce gradual apodization filter shapes making complex beam shaping possible. Uniform acoustic fields are important to ensure uniform treatment is obtained without the need for image-based guidance.

### Gel Sense Applications

The ability to sense effective acoustic coupling of the ultrasonic transducer to the patient is beneficial. The present technology may incorporate an inductively tuned design element that enables a high impedance resonance to occur when the transducer is in air (air coupled) and a low electrical impedance when the transducer is in contact with water, gel, or tissue, for example when coupled to a patient's skin through gel. The use of an inductor or other electrical element or circuit to resonate with an ideal λ/4 matching structure. further benefits the designer by decoupling the gel sensing impedance characteristics of the transducer from the actual acoustic layers, so that manufacturing tolerances can be compensated for simply by choosing a slightly different value inductor for example, to maximise the impedance maxima for a given acoustic stack. By incorporating this impedance response directly into the transducer, any LIPUS systems can simply measure the average current flowing though the transducer to determine if effective coupling ahs been achieved. Also, since the present technology can achieve in excess of 5 times the magnitude of the impedance while air coupled compared to when it is effectively coupled to the patient, it is also possible to identify partially coupled conditions accurately.

Several alternative embodiments and examples have been described and illustrated herein. The embodiments described above are intended to be exemplary only. A person skilled in the art would appreciate the features of the individual embodiments, and the possible combinations and variations of the components. A person skilled in the art would further appreciate that any of the embodiments could be provided in any combination with the other embodiments disclosed herein. The present examples and embodiments, therefore, are to be considered in all respects as illustrative and non-restrictive. Accordingly, while specific embodiments have been illustrated and described, numerous modifications come to mind without significantly departing from the scope defined in the current description.

1. An ultrasonic transducer, comprising:
a low-volume fraction piezoelectric composite disc having resonant properties:
at least one electrode in electrical contact with the low-volume fraction piezoelectric composite disc: and
an annular apodizing backing structure in acoustic contact with the low-volume fraction piezoelectric composite disc, the annular apodizing backing structure having:
an inner perimeter and a corresponding inner thickness:
an outer perimeter and a corresponding outer thickness; and
an inclined surface forming a substantially continuous slope extending from the inner perimeter to the outer perimeter, the inner thickness being smaller than the outer thickness,
wherein the annular apodizing backing structure is configured to change an apparent thickness of the low-volume fraction piezoelectric composite disc with respect to the resonant properties of the low-volume fraction piezoelectric composite disc, thereby allowing the ultrasonic transducer to generate an acoustic field comprising at least one substantially uniform near field portion.

2. The ultrasonic transducer of claim 1, wherein the low-volume fraction piezoelectric composite disc is in a 1 3 configuration.

3. The ultrasonic transducer of claim 2, wherein the low-volume fraction piezoelectric composite disc comprises 280 μm by 280 μm pillars distributed in a 2D matrix pattern having a pitch of about 480 μm in both lateral axes.

4. The ultrasonic transducer of any one of claims 1 to 3, wherein the low-volume fraction piezoelectric composite disc is configured to operate in a half-wave resonant mode at 1.5 MHz.

5. The ultrasonic transducer of any one of claims 1 to 4, wherein the low-volume fraction piezoelectric composite disc has an acoustic impedance included in a range extending from about 9 MR to about 13 MR.

6. The ultrasonic transducer of claim 5, wherein the acoustic impedance is about 11 MR.

7. The ultrasonic transducer of any one of claims 1 to 6, wherein the low-volume fraction piezoelectric composite disc has a thickness of about λ/2 at 1.5 MHz.

8. The ultrasonic transducer of any one of claims 1 to 7, wherein the low-volume fraction piezoelectric composite disc comprises a lead zirconate titanate material (PZT) based material.

9. The ultrasonic transducer of claim 8, wherein the PZT-based material is PZT 5H.

10. The ultrasonic transducer of claim 8 or 9, wherein the low-volume fraction piezoelectric composite disc comprises about 35% of PZT 5H and about 65% of a polymer matrix.

11. The ultrasonic transducer of claim 10 wherein the polymer matrix comprises epoxy filled with micro glass balloons and silicone particles.

12. The ultrasonic transducer of any one of claims 9 to 11, wherein the PZT 5H pillars have a first bar-mode longitudinal acoustic velocity and the polymer matrix has a second longitudinal acoustic velocity, the second longitudinal velocity being approximately 60% to 70% of the first longitudinal velocity.

13. The ultrasonic transducer of claim 12, wherein the first longitudinal bar-mode acoustic velocity is about 3850 m/s and the second longitudinal acoustic velocity is about 2515 m/s.

14. The ultrasonic transducer of any one of claims 1 to 13, further comprising a ring-shaped printed circuit board having an inner diameter, wherein:
the low-volume fraction piezoelectric composite disc has an outer diameter, the inner diameter of the ring-shaped printed circuit board being larger than the outside diameter of the low-volume fraction piezoelectric composite disc.

15. The ultrasonic transducer of claim 14, further comprising a housing, the housing being made from plastic.

16. The ultrasonic transducer of claim 15, wherein the housing comprises a matching layer in acoustic communication with the low-volume fraction piezoelectric composite disc.

17. The ultrasonic transducer of claim 16, wherein the matching layer has a thickness of about λ/4.

18. The ultrasonic transducer of claim 16 or 17, wherein the matching layer is integrally formed with the housing.

19. The ultrasonic transducer of any one of claims 16 to 18, wherein the matching layer has an acoustic impedance included in a range extending from about 2.1 MR to about 2.5 MR.

20. The ultrasonic transducer of claim 19, wherein the acoustic impedance is about 2.3 MR.

21. The ultrasonic transducer of any one of claims 1 to 15, further comprising a 2λ/3 acoustic layer in acoustic communication with the low-volume fraction piezoelectric composite disc.

22. The ultrasonic transducer of claim 21, wherein the 2λ/3 acoustic layer is made from nonyl plastic and has a thickness of about 953 μm.

23. The ultrasonic transducer of any one of claims 1 to 15, further comprising a 0.9λ acoustic layer, in acoustic communication with the low-volume fraction piezoelectric composite disc.

24. The ultrasonic transducer of claim 23, wherein the 0.9λ acoustic layer is made from nonyl plastic and has a thickness of about 1.28 mm.

25. The ultrasonic transducer of claim 14, wherein the ring-shaped printed circuit board comprises two opposed planar surfaces, each planar surface being made from copper.

26. The ultrasonic transducer of claim 25, wherein the ring-shaped printed circuit board is bonded with a perimeter of the low-volume fraction piezoelectric composite disc.

27. The ultrasonic transducer of any one of claims 1 to 20, further comprising an inductor connected in parallel with a piezocomposite of the low-volume fraction piezoelectric composite disc, the inductor being configured to resonate with the low-volume fraction piezoelectric composite disc, the annular apodizing backing structure, and the ¼ lambda acoustic matching layer, such that an impedance maximum is produced at about 1.5 MHz when a distal face of the transducer is air loaded.

28. The ultrasonic transducer of any one of claims 1 to 20, further comprising an inductor connected in series with a low-volume fraction piezoelectric composite disc, the inductor being configured to resonate with the low-volume fraction piezoelectric composite disc the annular apodizing backing structure, and the ¼ lambda matching layer, such that an impedance minimum is produced at about 1.5 MHz when a distal face of the transducer is air loaded.

29. The ultrasonic transducer of any one of claims 1 to 28, wherein the annular apodizing backing structure comprises Epotek 301 epoxy.

30. The ultrasonic transducer of any one of claims 1 to 29, wherein the annular apodizing backing structure has an acoustic impedance of about 2.8 MR.

31. The ultrasonic transducer of any one of claims 1 to 30, wherein the slope is comprised between 0 degrees and 30 degrees.

32. The ultrasonic transducer of claim 31, wherein the slope is about 14 degrees with respect to a top surface of the low-volume fraction piezoelectric composite disc.

33. The ultrasonic transducer of any one of claims 1 to 32, wherein the ultrasonic transducer is operable at a frequency of about 1.5 MHz.

34. The ultrasonic transducer of any one of claims 1 to 33, wherein the ultrasonic transducer is operable in a narrow bandwidth tone burst mode.

35. The ultrasonic transducer of claim 34, wherein the narrow bandwidth tone burst mode is a 20% duty cycle sinusoidal pulsed mode, preferably at a pulse repetition frequency of about 1 kHz.

36. The ultrasonic transducer of any one of claims 1 to 35, wherein the ultrasonic transducer has a beam non-uniformity ratio of less than 3.5.

37. The ultrasonic transducer of any one of claims 1 to 36, wherein the at least one substantially uniform near field portion exhibits less than 2 dB of ripples in a plane located at about 3 mm of an external surface of the ultrasonic transducer, when the ultrasonic transducer is operated at 1.5 MHz with a 20% pulsed transmit waveform.

38. A low-intensity pulsed ultrasound (LIPUS) treatment head having an operating frequency, the LIPUS treatment head comprising:
an acoustic stack, comprising:
a piezoelectric disc, the piezoelectric disc comprising a low-volume fraction piezoelectric composite disc, the low-volume fraction piezoelectric composite disc being configured to operate in a half-wave resonant mode at the operating frequency of the LIPUS treatment head; an
an annular apodizing backing structure in acoustic communication with the low-volume fraction piezoelectric composite disc, the annular apodizing backing structure having an inner perimeter and an outer perimeter, respectively having an inner thickness and an outer thickness, the inner thickness being smaller than the outer thickness, the annular apodizing backing structure being configured to change an apparent thickness of the low-volume fraction piezoelectric composite disc with respect to the resonant properties of the low-volume fraction piezoelectric composite disc, thereby allowing the LIPUS treatment head to generate an acoustic field comprising at least one substantially uniform near field portion;
at least one electrode in electrical communication with the low-volume fraction piezoelectric composite disc; and
a housing for supporting the acoustic stack and the at least one electrode.

39. The LIPUS treatment head of claim 38, wherein the low-volume fraction piezoelectric composite disc is in a 1 3 configuration.

40. The LIPUS treatment head of claim 39, wherein the low-volume fraction piezoelectric composite disc comprises 280 μm by 280 μm pillars distributed in a 2D matrix pattern having a pitch of about 480 μm in both lateral axes.

41. The LIPUS treatment head of any one of claims 38 to 40, wherein the operating frequency of the LIPUS treatment head is about 1.5 MHz.

42. The LIPUS treatment head of any one of claims 38 to 41, wherein the low-volume fraction piezoelectric composite disc has an acoustic impedance included in a range extending from about 9 MR to about 13 MR.

43. The LIPUS treatment head of claim 42, wherein the acoustic impedance is about 11 MR.

44. The LIPUS treatment head of any one of claims 38 to 43, wherein the low-volume fraction piezoelectric composite disc has a thickness of about λ/2 at 1.5 MHz.

45. The ultrasonic transducer of any one of claims 38 to 44, wherein the low-volume fraction piezoelectric composite disc comprises a lead zirconate titanate material (PZT) based material.

46. The ultrasonic transducer of claim 45, wherein the PZT-based material is PZT 5H.

47. The LIPUS treatment head of claim 45 or 46, wherein the low-volume fraction piezoelectric composite disc comprises about 35% of PZT 5H and about 65% of a polymer matrix.

48. The LIPUS treatment head of claim 47, wherein the polymer matrix comprises epoxy filled with micro glass balloons and silicone particles.

49. The LIPUS treatment head of any one of claims 46 to 48, wherein the PZT 5H pillars have a first bar-mode longitudinal acoustic velocity and the polymer matrix has a second longitudinal acoustic velocity, the second longitudinal velocity being approximately 60 to 70% of the first longitudinal velocity.

50. The LIPUS treatment head of claim 49, wherein the first longitudinal acoustic velocity is about 3850 m/s and the second longitudinal acoustic velocity is about 2515 m/s.

51. The LIPUS treatment head of any one of claims 38 to 50, further comprising a ring-shaped printed circuit board having an inner diameter, wherein:
the low-volume fraction piezoelectric composite disc has an outer diameter, the inner diameter of the ring-shaped printed circuit board being larger than the outside diameter of the low-volume fraction piezoelectric composite disc.

52. The LIPUS treatment head of any one of claims 38 to 51, wherein the housing is made from plastic.

53. The LIPUS treatment head of any one of claims 38 to 52, wherein the housing comprises a matching layer in acoustic communication with the low-volume fraction piezoelectric composite disc.

54. The LIPUS treatment head of claim 53, wherein the matching layer has a thickness of about λ/4.

55. The LIPUS treatment head of claim 53 or 54, wherein the matching layer is integrally formed with the housing.

56. The LIPUS treatment head of any one of claims 53 to 55, wherein the matching layer has acoustic impedance included in a range extending from about 2.1 MR to about 2.5 MR.

57. The LIPUS treatment head of claim 56, wherein the acoustic impedance is about 2.3 MR.

58. The LIPUS treatment head of any one of claims 38 to 57, further comprising a 2λ/3 acoustic layer in acoustic communication with the low-volume fraction piezoelectric composite disc.

59. The LIPUS treatment head of claim 58, wherein the 2λ/3 acoustic layer is made from nonyl plastic and has a thickness of about 953 μm.

60. The LIPUS treatment head of claims 38 to 57, further comprising a 0.9λ acoustic layer, in acoustic communication with the low-volume fraction piezoelectric composite disc.

61. The LIPUS treatment head of claim 60, wherein the 0.9λ acoustic layer is made from nonyl plastic and has a thickness of about 1.28 mm.

62. The LIPUS treatment head of claim 51, wherein the ring-shaped printed circuit board comprises two opposed planar surfaces, each planar surface being made from copper.

63. The LIPUS treatment head of claim 62, wherein the ring-shaped printed circuit board is bonded with a perimeter of the low-volume fraction piezoelectric composite disc.

64. The LIPUS treatment head of any one of claims 38 to 57, further comprising an inductor connected in parallel with a piezocomposite of the low-volume fraction piezoelectric composite disc, the inductor being configured to resonate with the low-volume fraction piezoelectric composite disc, the annular apodizing backing structure, and the ¼ lambda matching layer, such that an impedance maximum is produced at approximately 1.5 MHz when a distal face of the transducer is air loaded.

65. The LIPUS treatment head of any one of claims 38 to 57, further comprising an inductor connected in series with a piezocomposite of the low-volume fraction piezoelectric composite disc, the inductor being configured to resonate with the low-volume fraction piezoelectric composite disc, the annular apodizing backing structure, and the ¼ lambda matching layer, such that an impedance minimum is produced at approximately 1.5 MHz when the distal face of the transducer is air loaded.

66. The LIPUS treatment head of any one of claims 38 to 65, wherein the annular apodizing backing structure comprises Epotek 301 epoxy.

67. The LIPUS treatment head of any one of claims 38 to 66, wherein the annular apodizing backing structure has an acoustic impedance of about 2.8 MR.

68. The LIPUS treatment head of any one of claims 38 to 67, wherein the slope is comprised between 0 to 30 degrees.

69. The LIPUS treatment head of claim 68, wherein the slope is about 14 degrees with respect to a top surface of the low-volume fraction piezoelectric composite disc.

70. The LIPUS treatment head of any one of claims 38 to 69, wherein the operating frequency of LIPUS treatment head is about 1.5 MHz.

71. The LIPUS treatment head of any one of claims 38 to 70, wherein the LIPUS treatment head is operable in a narrow bandwidth tone burst mode.

72. The LIPUS treatment head of claim 71, wherein the narrow bandwidth tone burst mode is a 20% duty cycle sinusoidal pulsed mode.

73. The LIPUS treatment head of any one of claims 38 to 72, wherein the LIPUS treatment head has beam non-uniformity ratio of less than 3.5.

74. The LIPUS treatment head of any one of claims 38 to 73, wherein the at least one substantially uniform near field portion exhibits less than 2 dB of ripple in a plane located at about 3 mm of an external surface of the LIPUS treatment head, when the LIPUS treatment head is operated at 1.5 MHz with a 20% pulsed transmit waveform.

75. An apodizing wedge structure for a low-intensity pulsed ultrasound (LIPUS) treatment head, the LIPUS treatment head comprising a low-volume fraction piezoelectric composite disc, the apodizing wedge structure comprising:
an annular body for contacting a surface of the low-volume fraction piezoelectric composite disc, the annular body comprising an inner perimeter having a corresponding inner thickness and an outer perimeter having a corresponding outer thickness,
wherein the annular body comprises an inclined surface forming a substantially continuous slope extending from the inner perimeter to the outer perimeter, the inner thickness being smaller than the outer thickness, the apodizing wedge being configured to change an apparent thickness of the low-volume fraction piezoelectric composite disc with respect to resonant properties of the low-volume fraction piezoelectric composite disc when the apodizing wedge structure is in acoustic communication with the low-volume fraction piezoelectric composite disc, thereby allowing the LIPUS treatment head to generate an acoustic field comprising at least one substantially uniform near field portion.

76. The apodizing wedge structure of claim 75, wherein the annular body is made from Epotek 301 epoxy.

77. The apodizing wedge structure of claim 75 or 76, wherein the annular body has an acoustic impedance of about 2.8 MR.

78. The apodizing wedge structure of any one of claims 75 to 77, wherein the slope is comprised between 0 to 30 degrees.

79. The apodizing wedge structure of claim 78, wherein the slope is about 14 degrees with respect to a top surface of the low-volume fraction piezoelectric composite disc.

80. The apodizing wedge structure of any one of claims 75 to 79, wherein the LIPUS treatment head is operable at a frequency of about 1.5 MHz.

81. The apodizing wedge structure of any one of claims 75 to 80, wherein the LIPUS treatment head is operable in a narrow bandwidth tone burst mode.

82. The apodizing wedge structure of claim 81, wherein the narrow bandwidth tone burst mode is a 20% duty cycle sinusoidal pulsed mode.

83. The apodizing wedge structure of any one of claims 75 to 82, wherein the LIPUS treatment head has a beam non-uniformity ratio of less than 3.5.

84. The apodizing wedge structure of any one of claims 75 to 83, wherein the at least one substantially uniform near field portion exhibits less than 2 dB of ripples in a plane located at about 3 mm of an external surface of the LIPUS treatment head, when the LIPUS treatment head is operated at 1.5 MHz with a 20% pulsed transmit waveform.

85. A backing structure for a low-intensity pulsed ultrasound (LIPUS) treatment head, the LIPUS treatment head comprising a low-volume fraction piezoelectric composite element, the backing structure comprising:
a body for contacting a surface of the low-volume fraction piezoelectric composite element, such that when the body contacts the low-volume fraction piezoelectric composite element, destructive interference is produced within the backing structure and the low-volume fraction piezoelectric component, thereby shaping an acoustic field generated by the LIPUS treatment head, the destructive interference being dependent on a thickness of the backing structure.

86. The backing structure of claim 85, wherein the destructive interference results in a maximal attenuation at approximately λ/4 or odd multiples thereof.

87. The backing structure of claim 85 or 86, wherein the body is made from Epotek 301 epoxy.

88. The backing structure of any one of claims 85 to 87, wherein the LIPUS treatment head is operable at a frequency of about 1.5 MHz.

89. The backing structure of any one of claims 85 to 88, wherein the LIPUS treatment head is operable in a narrow bandwidth tone burst mode.

90. The backing structure of claim 89, wherein the narrow bandwidth tone burst mode is a 20% duty cycle sinusoidal pulsed mode.

91. The backing structure of any one of claims 85 to 90, wherein the LIPUS treatment head has a beam non-uniformity ratio of less than 3.5.

92. The backing structure of any one of claims 85 to 91, wherein the acoustic field comprises at least one substantially uniform near field portion, said at least one substantially uniform near field portion exhibiting less than 2 dB of ripples in a plane located at about 3 mm of an external surface of the LIPUS treatment head, when the LIPUS treatment head is operated at 1.5 MHz with a 20% pulsed transmit waveform.

93. A method of apodizing an acoustic field, the method comprising:
operating an ultrasonic transducer to generate the acoustic field, the ultrasonic transducer comprising a low-volume fraction piezoelectric composite disc, the low-volume fraction piezoelectric composite having resonant properties; and
conditioning the acoustic field with an annular apodizing backing structure to generate an apodized acoustic field, the apodized acoustic field comprising at least one substantially uniform near field portion, the annular apodizing backing structure being in acoustic contact with the low-volume fraction piezoelectric composite disc, the annular apodizing backing structure having:
an inner perimeter and a corresponding inner thickness:
an outer perimeter and a corresponding outer thickness: and
an inclined surface forming a substantially continuous slope extending from the inner perimeter to the outer perimeter, the inner thickness being smaller than the outer thickness,
wherein the annular apodizing backing structure is configured to change an apparent thickness of the low-volume fraction piezoelectric composite disc with respect to the resonant properties of the low-volume fraction piezoelectric composite disc.

94. The method of claim 93, wherein:
the ultrasonic transducer is operated at 1.5 MHz with a 20% pulsed transmit waveform; and
the at least one substantially uniform near field portion exhibits less than 2 dB of ripples in a plane located at about 3 mm of an external surface of the ultrasonic transducer.

95. The method of claim 93 or 94, wherein the low-volume fraction piezoelectric composite disc is in a 1 3 configuration.

96. The method of any one of claims 93 to 95, wherein the low-volume fraction piezoelectric composite disc comprises 280 μm by 280 μm pillars distributed in a 2D matrix pattern having a pitch of about 480 μm in both lateral axes.

97. A method for generating an acoustic field with a low-intensity pulsed ultrasound (LIPUS) treatment head having an operating frequency, the method comprising:
operating the LIPUS treatment to generate the acoustic field, the LIPUS treatment head comprising:
an acoustic stack, the acoustic stack comprising:
a piezoelectric disc, the piezoelectric disc comprising a low-volume fraction piezoelectric composite disc, the low-volume fraction piezoelectric composite disc being configured to operate in a half-wave resonant mode at the operating frequency of the LIPUS treatment head; and
an annular apodizing backing structure in acoustic communication with the low-volume fraction piezoelectric composite disc, the annular apodizing backing structure having an inner perimeter and an outer perimeter, respectively having an inner thickness and an outer thickness, the inner thickness being smaller than the outer thickness;
at least one electrode in electrical communication with the low-volume fraction piezoelectric composite disc; and
a housing for supporting the acoustic stack and the at least one electrode; and
conditioning the acoustic field with the annular apodizing backing structure to generate an apodized acoustic field, the apodized acoustic field comprising at least one substantially uniform near field portion.

98. The method of claim 97, wherein:
the ultrasonic transducer is operated at 1.5 MHz with a 20% pulsed transmit waveform; and
the at least one substantially uniform near field portion exhibits less than 2 dB of ripples in a plane located at about 3 mm of an external surface of the ultrasonic transducer.

99. The method of claim 97 or 98, wherein the low-volume fraction piezoelectric composite disc is in a 1 3 configuration.

100. The method of any one of claims 97 to 99, wherein the low-volume fraction piezoelectric composite disc comprises 280 μm by 280 μm pillars distributed in a 2D matrix pattern having a pitch of about 480 μm in both lateral axes.
